(19) United States
(12) Patent Application Publication
(10) Pub. No.: US 2007/0281363 A1
(43) Pub. Date: Dec. 6, 2007

(54) LUMINESCENT COMPOUNDS

(75) Inventors: Leonid D. Patsenker, Kharkov (UA); Ewald A. Terpetschnig, Urbana, IL (US); Irina A. Fedyunyaeva, Kharkov (UA); Olga N. Kolosova, Kharkov (UA); Alexey Klochko, Kharkov (UA)

Correspondence Address: KOLISCH HARTWELL, P.C., 520 SW YAMHILL STREET, Suite 200, PORTLAND, OR, 97204, US

(73) Assignee: Ewald A. Terpetschnig, Urbana, IL (US)

(21) Appl. No.: 11/734,748

(22) Filed: Apr. 12, 2007

Related U.S. Application Data
(60) Provisional application No. 60/792,130, filed on Apr. 13, 2006.

(51) Int. Cl. G01N 21/00; C07D 217/00; C07D 239/00; C07D 245/00; C07D 263/62; C07D 401/00; C07D 417/00; C07D 487/02; C07H 21/04; C07K 14/00; C07K 2/00

(52) U.S. Cl. 436/164; 530/300; 530/402; 536/23.1; 540/460; 544/249; 544/300; 546/150; 548/159; 548/220; 548/433

(57) ABSTRACT

Reporter compounds based on cyanine dyes, among others, including reactive intermediates used to synthesize the reporter compounds, and methods of synthesizing and using the reporter compounds, among others, where the reporter compounds relate generally to the following structure:

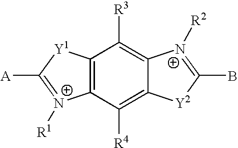

### CROSS-REFERENCES TO PRIORITY APPLICATIONS

This application is based upon and claims the benefit under 35 U.S.C. § 119(e) of U.S. Provisional Patent Application Ser. No. 60/792,130, filed Apr. 13, 2006, which is incorporated herein by reference in its entirety for all purposes.

### CROSS-REFERENCES TO RELATED MATERIALS

This application incorporates by reference in their entirety for all purposes all patents, patent applications (published, pending, and/or abandoned), and other patent and nonpatent references cited anywhere in this application. The cross-referenced materials include but are not limited to the following publications: Richard P. Haugland, HANDBOOK OF FLUORESCENT PROBES AND RESEARCH CHEMICALS (6^{th} ed. 1996); JOSEPH R. LAKOWICZ, PRINCIPLES OF FLUORESCENCE SPECTROSCOPY (2^{nd} Ed. 1999); RICHARD J. LEWIS, SR., HAWLEY'S CONDENSED CHEMICAL DICTIONARY (12^{th} ed. 1993).

### TECHNICAL FIELD

The present disclosure relates to compounds based on cyanines, squaraines and styryl, among others. More particularly, the disclosure relates to compounds based on pyrroloindoles, among others, that may be useful as both non-fluorescent labels and luminescent reporters.

### BACKGROUND

Colorimetric and/or luminescent compounds may offer researchers the opportunity to use color and light to analyze samples, investigate reactions, and perform assays, either qualitatively or quantitatively. Generally, brighter, more photostable reporters may permit faster, more sensitive, and more selective methods to be utilized in such research.

While a calorimetric compound absorbs light, and may be detected by that absorbance, a luminescent compound, or luminophore, is a compound that emits light. A luminescence method, in turn, is a method that involves detecting light emitted by a luminophore, and using properties of that light to understand properties of the luminophore and its environment. Luminescence methods may be based on chemiluminescence and/or photoluminescence, among others, and may be used in spectroscopy, microscopy, immunoassays, and hybridization assays, among others.

Photoluminescence is a particular type of luminescence that involves the absorption and subsequent re-emission of light. In photoluminescence, a luminophore is excited from a low-energy ground state into a higher-energy excited state by the absorption of a photon of light. The energy associated with this transition is subsequently lost through one or more of several mechanisms, including production of a photon through fluorescence or phosphorescence.

Photoluminescence may be characterized by a number of parameters, including extinction coefficient, excitation and emission spectrum, Stokes' shift, luminescence lifetime, and quantum yield. An extinction coefficient is a wavelength-dependent measure of the absorbing power of a luminophore. An excitation spectrum is the dependence of emission intensity upon the excitation wavelength, measured at a single constant emission wavelength. An emission spectrum is the wavelength distribution of the emission, measured after excitation with a single constant excitation wavelength. A Stokes' shift is the difference in wavelengths between the maximum of the emission spectrum and the maximum of the absorption spectrum. A luminescence lifetime is the average time that a luminophore spends in the excited state prior to returning to the ground state. A quantum yield is the ratio of the number of photons emitted to the number of photons absorbed by a luminophore.

Luminescence methods may be influenced by extinction coefficient, excitation and emission spectra, Stokes' shift, and quantum yield, among others, and may involve characterizing fluorescence intensity, fluorescence polarization (FP), fluorescence resonance energy transfer (FRET), fluorescence lifetime (FLT), total internal reflection fluorescence (TIRF), fluorescence correlation spectroscopy (FCS), fluorescence recovery after photobleaching (FRAP), and their phosphorescence analogs, among others.

Luminescence methods have several significant potential strengths. First, luminescence methods may be very sensitive, because modern detectors, such as photomultiplier tubes (PMTs) and charge-coupled devices (CCDs), can detect very low levels of light. Second, luminescence methods may be very selective, because the luminescence signal may come almost exclusively from the luminophore.

Despite these potential strengths, luminescence methods may suffer from a number of shortcomings, at least some of which relate to the luminophore. For example, the luminophore may have an extinction coefficient and/or quantum yield that is too low to permit detection of an adequate amount of light. The luminophore also may have a Stokes' shift that is too small to permit detection of emission light without significant detection of excitation light. The luminophore also may have an excitation spectrum that does not permit it to be excited by wavelength-limited light sources, such as common lasers and arc lamps. The luminophore also may be unstable, so that it is readily bleached and rendered nonluminescent. The luminophore also may have an excitation and/or emission spectrum that overlaps with the well-known autoluminescence of biological and other samples; such autoluminescence is particularly significant at wavelengths below about 600 nm. The luminophore also may be expensive, especially if it is difficult to manufacture.

### SUMMARY

The present disclosure provides reporter compounds based on cyanines and squaraines, among others, reactive intermediates used to synthesize the reporter compounds, and methods of synthesizing and using the reporter compounds, among others.

The fluorescent or non-fluorescent compounds relate generally to the following structure:

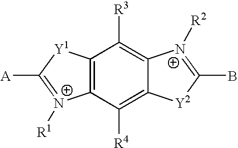

wherein

each A is selected from a group consisting of H, alkyl, alkenyl, alkynyl, aryl, halogen, sulfo, carboxy, formylmethylene, phosphate, amino, sulfate, phosphonate, cyano, nitro, azido, reactive aliphatic and reactive aromatic groups and W¹, W², W³, W⁴, W⁵;

B is selected from the group consisting of W¹, W², W³, W⁴, W⁵; wherein W¹, W², W³, W⁴, W⁵ have the respective formulae

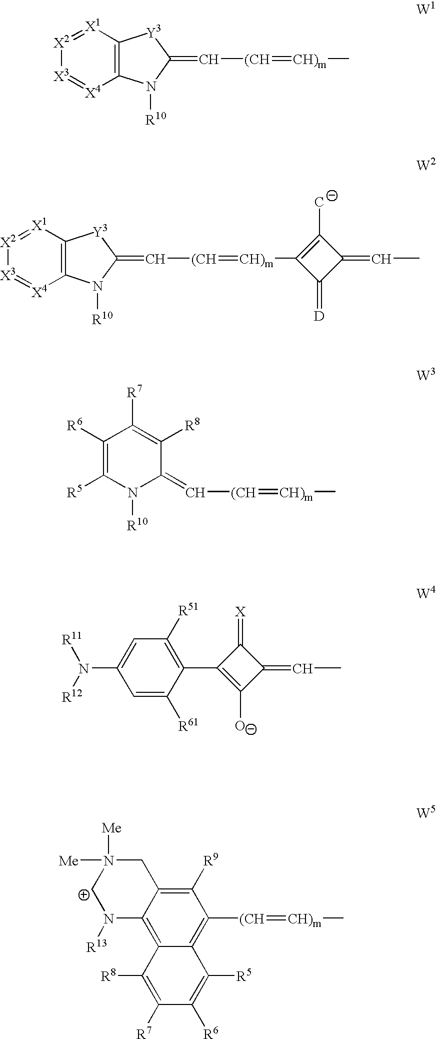

each R¹ and R² is independently selected from H, aliphatic groups, alicyclic groups, alkylaryl groups, aromatic groups, -L-S_{c}, -L-R^{x}, -L-R^{±} among others.

each of X¹, X², X³, and X⁴ are independently selected from the group consisting of N, NR^{ι}, O, S, and C—R^{τ} among others.

R^{τ}, R³, R⁴, R⁵, R⁶, R⁷, R⁸ and R⁹ is hydrogen, -L-S_{c}, -L-R^{x}, -L-R^{±}, —R^{x}, —R^{±} among others.

Y¹, Y² and Y³ are each independently selected from O, S, Se, N—R^{d}, CR^{e}═CR^{f} and C(Rⁱ)(R^{j}) among others;

R¹¹ and R¹² are independently H, alkyl, aryl, -L-S_{c}, -L-R^{x}, -L-R^{±}, or taken in combination, form a cyclic or heterocyclic ring structure which is optionally substituted by -L-S_{c}, -L-R^{x} or -L-R^{±};

R⁵¹ and R⁶¹ are independently H, OH, O-alkyl, NH-alkyl, NH-aryl;

m is 0, 1, 2 or 3.

The components R¹-R¹², m, X¹, X², X³, X⁴, and Y¹, Y², Y³ are defined in detail in the Detailed Description. The compound may include a reactive group and/or a carrier. Alternatively, or in addition the substituents may be chosen so that the compound is photoluminescent, or not fluorescent at all.

The methods relate generally to the synthesis and/or use of reporter compounds, especially those described above.

The nature of the disclosed compositions will be understood more readily after consideration of the drawing, chemical structures, and detailed description that follow.

### BRIEF DESCRIPTION OF THE DRAWINGS

FIG. 1 shows the absorption spectrum of compound 5b in water.

### ABBREVIATIONS

The following abbreviations, among others, may be used in this application:

| Abbreviation | Definition |
|---|---|
| abs | Absorption |
| BSA | bovine serum albumin |
| Bu | Butyl |
| DCC | dicyclohexylcarbodiimide |
| DMF | dimethylformamide |
| DMSO | dimethylsulfoxide |
| DIP | dye-to-protein ratio |
| Et | ethyl |
| fl | Fluorescence |
| g | grams |
| h | hours |
| HSA | human serum albumin |
| L | liters |
| Lit. | Literature |
| m | milli (10⁻³) |
| M | molar |
| Me | methyl |
| mol | moles |
| M.P. | melting point |
| n | nano (10⁻⁹) |
| NHS | N-hydroxysuccinimide |
| NIR | near infrared region |
| PB | Phosphate buffer |
| Prop | propyl |
| μ | micro (10⁻⁶) |

### DETAILED DESCRIPTION

The present disclosure relates generally to dyes (fluorescent and non-fluorescent) and their synthetic precursors, and to methods of synthesizing and using such compounds. These compounds may be useful in both free and conjugated forms, as probes, labels, and/or indicators. This usefulness may reflect in part enhancement of one or more of the following: extinction coefficient, quantum yield, Stokes' shift, and photostability. This usefulness also may reflect absorption or excitation and emission spectra in relatively inaccessible regions of the spectrum, including the red and near infrared.

The remaining discussion includes (1) an overview of structures, (2) an overview of synthetic methods, and (3) a series of illustrative examples.

Overview of Structures

The reporter compounds and their synthetic precursors may be generally described by the following structure:

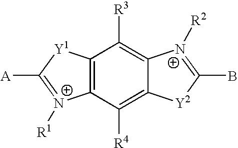

Here, A is selected from the group consisting of H, alkyl, alkenyl, alkynyl, aryl, halogen, sulfo, carboxy, formylmethylene, phosphate, amino, sulfate, phosphonate, cyano, nitro, azido, reactive aliphatic and reactive aromatic groups and W¹, W², W³, W⁴, W⁵;

B is selected from the group consisting of W¹, W², W³, W⁴, W⁵; wherein W¹, W², W³, W⁴, W⁵ have the respective formulae:

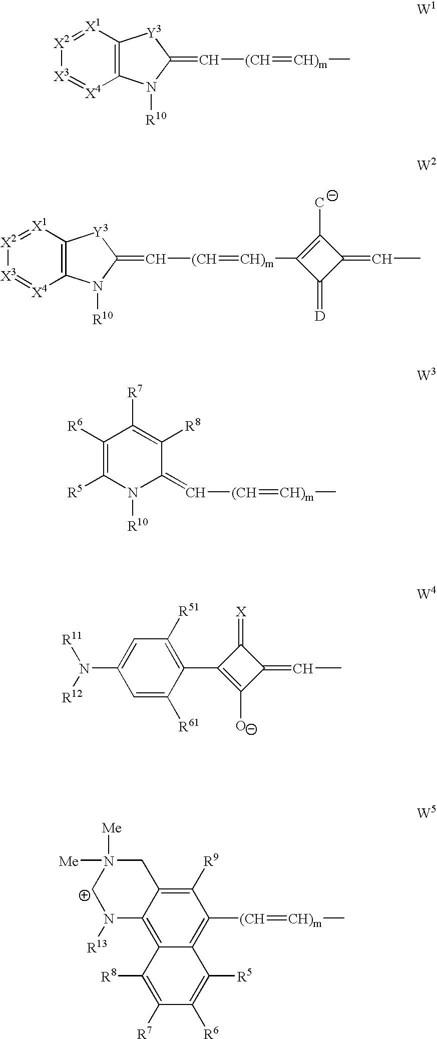

each R¹, R² and R¹⁰ is independently selected from H, aliphatic groups, alicyclic groups, alkylaryl groups, aromatic groups, -L-S_{c}, -L-R^{x}, -L-R^{±}, —CH₂—CONH—SO₂-Me; each aliphatic residue may incorporate up to six heteroatoms selected from N, O, S, and can be substituted one or more times by F, Cl, Br, I, hydroxy, alkoxy, carboxy, sulfo, phosphate, amino, sulfate, phosphonate, cyano, nitro, azido, alkyl-amino, dialkyl-amino or trialkylammonium;

L is a covalent linkage that is linear or branched, cyclic or heterocyclic, saturated or unsaturated, having 1-20 nonhydrogen atoms from the group of C, N, P, O and S, in such a way that the linkage contains any combination of ether, thioether, amine, ester, amide bonds; single, double, triple or aromatic carbon-carbon bonds; or carbon-sulfur bonds, carbon-nitrogen bonds, phosphorus-sulfur, nitrogen-nitrogen, nitrogen-oxygen or nitrogen-platinum bonds, or aromatic or heteroaromatic bonds;

R^{x} is a reactive group;

S_{c} is a conjugated substance;

R^{±} is an ionic group;

each of X¹, X², X³, and X⁴ are independently selected from the group consisting of N, NR^{ι}, O, S, and C—R^{τ}, where R^{ι} is hydrogen, alkyl, arylalkyl and aryl groups, -L-S_{c}, -L-R^{x}, -L-R^{±}, —CH₂—CONH—SO₂-Me, where each aliphatic residue may incorporate up to six heteroatoms selected from N, O, S, and can be substituted one or more times by F, Cl, Br, I, hydroxy, alkoxy, carboxy, sulfo, phosphate, amino, sulfate, phosphonate, cyano, nitro, azido, alkyl-amino, dialkyl-amino or trialkylammonium;

R^{τ}, R³, R⁴, R⁵, R⁶, R⁷, R⁸ and R⁹ is hydrogen, -L-S_{c}, -L-R^{x}, -L-R^{±}, —R^{x}, —R^{±}, —CH₂—CONH—SO₂-Me, amino, alkylamino, dialkylamino, trialkylammonium, sulfo, carboxy, nitro, cyano, azido, trifluoromethyl, alkoxy, halogen, carboxy, hydroxy, phosphate, is sulfate or an aliphatic, alicyclic, or aromatic group among others;

adjacent R^{ι}, R^{τ}, R⁵, R⁶, R⁷ and R⁸ substituents, taken in combination, form a fused aromatic or heterocyclic ring that is itself optionally further substituted by H, alkyl, aryl, cycloalkyl, L-S_{c}, L-R^{x}, L-R^{±}, —R^{x} or —R^{±}; and

Y¹, Y² and Y³ are each independently selected from O, S, Se, N—R^{d}, CR^{e}═CR^{f} and C(Rⁱ)(R^{j}), wherein R^{d} is selected from the group consisting of H, aliphatic groups, alicyclic groups, aromatic groups, -L-S_{c}, -L-R^{x}, -L-R^{±}, —CH₂—CONH—SO₂-Me; and R^{e}, R^{f}, Rⁱ and R^{j} are selected from the group consisting of H, aliphatic groups, alicyclic groups, aromatic groups, -L-S_{c}, -L-R^{x}, -L-R^{±}, —R^{x}, —R^{±}, —CH₂—CONH—SO₂-Me, —COOH, —CN, —OH, —SO₃H, —PO₃H₂, —O—PO₃H₂, —PO₃R₂^{m}, —O—PO₃R₂^{m}, —CONHR^{m}, —CONH₂, COO—NHS and COO—R^{m}; R^{m} is selected from a group consisting of aliphatic groups, and aromatic substituents among others; Rⁱ and R^{j} taken in combination form a ring-system that is optionally further substituted by one or more reactive or ionic substituents;

D when present and neutral, is independently selected from the group consisting of ═O, ═S, ═Se, ═Te, ═N—R^{a}, and ═C(R^{b})(R^{c});

C when present and negatively charged, is independently selected from the group consisting of —O⁻, —S⁻, —Se⁻, —Te⁻, —(N—R^{a})⁻, and —(C(R^{b})(R^{c}))⁻;

each R^{a} may be independently selected from the group consisting of H, aliphatic, aromatic, alicyclic, aryl-alkyl, linked carriers, reactive and reactive aliphatic substituents, —COOH, —CN, —OH, —SO₃H, —SO₃R^{m}, —PO₃H₂, —O—PO₃H₂, —PO₃R₂^{m}, —O—PO₃R₂^{m}, —CONHR^{m}, —CONH₂, COO—NHS and COO—R^{m}; each R^{b} and R^{c} may be independently selected from the group consisting of H, aliphatic, aromatic, alicyclic, aryl-alkyl, -L-S_{c}, -L-R^{x}, -L-R^{±}, —COOH, —CN, —OH, —SO₃H, —PO₃H₂ among others; R^{m} is selected from a group consisting of aliphatic groups, and aromatic substituents among others;

or R^{b} and R^{c}, taken in combination, form a cyclic or heterocyclic ring structure which is optionally substituted by -L-S_{c}, L-R^{x} or -L-R^{±};

R¹¹, R¹² and R¹³ are independently H, alkyl, aryl, -L-S_{c}, -L-R^{x}, -L-R^{±} among others;

R⁵¹ and R⁶¹ are independently H, OH, O-alkyl, NH-alkyl, NH-aryl;

m is 0, 1, 2 or 3;

The substituents on the substituted rings may be chosen quite broadly, and may include the various components listed above, among others.

### Reporter Compounds

Where the reporter compound is a calorimetric dye and/or a photoluminescent compound, A and B are typically chosen from W¹-W⁵; W¹-W⁵ typically are present in any order. A is in many cases represented by CH₃ or a substituted alkyl residue.

The reporter compounds may be non-fluorescent calorimetric dyes, useful as stains and for calorimetric detection but in particular as non-fluorescent energy transfer acceptors in FRET-based applications. Alternatively or in addition, the reporter compounds may be photoluminescent, particularly fluorescent, and may have utility in photoluminescence assays and methods, as discussed above.

Synthetic Precursors.

A number of synthetic precursors are described in Example 1.

Reactive Groups (R^{x}).

The substituents on these compounds may include one or more reactive groups, where a reactive group generally is a group capable of forming a covalent attachment with another molecule or substrate. Such other molecules or substrates may include proteins, carbohydrates, nucleic acids, and plastics, among others. Reactive groups (R^{x}) vary in their specificity, and may preferentially react with particular functional groups and molecule types. Thus, reactive compounds generally include reactive groups chosen preferentially to react with functional groups found on the molecule or substrate with which the reactive compound is intended to react.

The compounds of the present disclosure are optionally substituted, either directly or via a substituent, by one or more chemically reactive functional groups that may be useful for covalently attaching the compound to a desired substance. Each reactive group R^{x}, may be bound to the compound directly by a single covalent bond (—R^{x}), or may be attached via a covalent spacer or linkage, -L-, and may be depicted as -L-R^{x}.

The reactive group (—R^{x}) of the present disclosure may be selected from the following functional groups, among others: activated carboxylic esters, acyl azides, acyl halides, acyl halides, acyl nitriles, acyl nitriles, aldehydes, ketones, alkyl halides, alkyl sulfonates, anhydrides, aryl halides, azindines, boronates, carboxylic acids, carbodiimides, diazoalkanes, epoxides, haloacetamides, halotriazines, imido esters, isocyanates, isothiocyanates, maleimides, phosphoramidites, silyl halides, sulfonate esters, and sulfonyl halides.

The following reactive functional groups (—R^{x}), among others, may be particularly useful for the preparation of labeled molecules or substances, and are therefore suitable reactive functional groups for the purposes of the reporter compounds:
a) N-hydroxysuccinimide esters, isothiocyanates, and sulfonylchlorides, which form stable covalent bonds with amines, including amines in proteins and amine-modified nucleic acids;
b) Iodoacetamides and maleimides, which form covalent bonds with thiol-functions, as in proteins;
c) Carboxyl functions and various derivatives, including N-hydroxybenztriazole esters, thioesters, p-nitrophenyl esters, alkyl, alkenyl, alkynyl, and aromatic esters, and acyl imidazoles;
d) Alkylhalides, including iodoacetamides and chloroacetamides;
e) Hydroxyl groups, which can be converted into esters, ethers, and aldehydes;
f) Aldehydes and ketones and various derivatives, including hydrazones, oximes, and semicarbozones;
g) Isocyanates, which may react with amines;
h) Activated C═C double-bond-containing groups, which may react in a Diels-Alder reaction to form stable ring systems under mild conditions;
i) Thiol groups, which may form disulfide bonds and react with alkylhalides (such as iodoacetamide);
j) Alkenes, which can undergo a Michael addition with thiols, e.g., maleimide reactions with thiols;
k) Phosphoramidites, which can be used for direct labeling of nucleosides, nucleotides, and oligonucleotides, including primers on solid or semi-solid supports;
l) Primary amines that may be coupled to variety of groups including carboxyl, aldehydes, ketones, and acid chlorides, among others; and
m) Boronic acid derivatives that may react with sugars.
   R Groups

The R moieties associated with the various substituents of Z may include any of a number of groups, as described above, including but not limited to aliphatic groups, alicyclic groups, aromatic groups, and heterocyclic rings, as well as substituted versions thereof.

Aliphatic groups may include groups of organic compounds characterized by straight- or branched-chain arrangement of the constituent carbon atoms. Aliphatic hydrocarbons comprise three subgroups: (1) paraffins (alkanes), which are saturated and comparatively unreactive; (2) olefins (alkenes or alkadienes), which are unsaturated and quite reactive; and (3) acetylenes (alkynes), which contain a triple bond and are highly reactive. In complex structures, the chains may be branched or cross-linked and may contain one or more heteroatoms (such as polyethers and polyamines, among others).

As used herein, “alicyclic groups” include hydrocarbon substituents that incorporate closed rings. Alicyclic substituents may include rings in boat conformations, chair conformations, or resemble bird cages. Most alicyclic groups are derived from petroleum or coal tar, and many can be synthesized by various methods. Alicyclic groups may optionally include heteroalicyclic groups, that include one or more heteroatoms, typically nitrogen, oxygen, or sulfur. These compounds have properties resembling those of aliphatics and should not be confused with aromatic compounds having the hexagonal benzene ring. Alicyclics may comprise three subgroups: (1) cycloparaffins (saturated), (2) cycloolefins (unsaturated with two or more double bonds), and (3) cycloacetylenes (cyclynes) with a triple bond. The best-known cycloparaffins (sometimes called naphthenes) are cyclopropane, cyclohexane, and cyclopentane; typical of the cycloolefins are cyclopentadiene and cyclooctatetraene. Most alicyclics are derived from petroleum or coal tar, and many can be synthesized by various methods.

Aromatic groups may include groups of unsaturated cyclic hydrocarbons containing one or more rings. A typical aromatic group is benzene, which has a 6-carbon ring formally containing three double bonds in a delocalized ring system. Aromatic groups may be highly reactive and chemically versatile. Most aromatics are derived from petroleum and coal tar. Heterocyclic rings include closed-ring structures, usually of either 5 or 6 members, in which one or more of the atoms in the ring is an element other than carbon, e.g., sulfur, nitrogen, etc. Examples include pyridine, pyrrole, furan, thiophene, and purine. Some 5-membered heterocyclic compounds exhibit aromaticity, such as furans and thiophenes, among others, and are analogous to aromatic compounds in reactivity and properties.

Any substituent of the compounds of the present disclosure, including any aliphatic, alicyclic, or aromatic group, may be further substituted one or more times by any of a variety of substituents, including without limitation, F, Cl, Br, I, carboxylic acid, sulfonic acid, CN, nitro, hydroxy, phosphate, phosphonate, sulfate, cyano, azido, amine, alkyl, alkoxy, trialkylammonium or aryl. Aliphatic residues can incorporate up to six heteroatoms selected from N, O, S. Alkyl substituents include hydrocarbon chains having 1-22 carbons, more typically having 1-6 carbons, sometimes called “lower alkyl”.

As described in WO01/11370, sulfonamide groups such as —(CH₂)ₙ—SO₂—NH—SO₂—R, —(CH₂)ₙ—CONH—SO₂—R, —(CH₂)ₙ—SO₂—NH—CO—R, and —(CH₂)ₙ—SO₂NH—SO₃H, where R is aryl or alkyl and n=1-6, can be used to reduce the aggregation tendency and have positive effects on the photophysical properties of cyanines and related dyes, in particular when these functional groups are directly associated with the benzazole ring in position 1 (the nitrogen atom in the azole ring).

Where a substituent is further substituted by a functional group R^{±} that is ionically charged, such as for example a carboxylic acid, sulfonic acid, phosphoric acid, phosphonate or a quaternary ammonium group, the ionic substituent R^{±} may serve to increase the overall hydrophilicity of the compound.

As used herein, functional groups such as “carboxylic acid,” “sulfonic acid,” and “phosphoric acid” include the free acid moiety as well as the corresponding metal salts of the acid moiety, and any of a variety of esters or amides of the acid moiety, including without limitation alkyl esters, aryl esters, and esters that may be cleavable by intracellular esterase enzymes, such as alpha-acyloxyalkyl ester (for example acetoxymethyl esters, among others).

The compounds of the present disclosure are optionally further substituted by a reactive functional group R^{x}, or a conjugated substance S_{c}, as described below.

The compounds of the present disclosure may be depicted in structural descriptions as possessing an overall charge, it is to be understood that the compounds depicted include an appropriate counter ion or counter ions to balance the formal charge present on the compound. Further, the exchange of counter ions is well known in the art and readily accomplished by a variety of methods, including ion-exchange chromatography and selective precipitation, among others.

Carriers and Conjugated Substances S_{c}

The reporter compounds of the present disclosure, including synthetic precursor compounds, may be covalently or noncovalently associated with one or more substances. Covalent association may occur through various mechanisms, including a reactive functional group as described above, and may involve a covalent linkage, -L-, separating the compound or precursor from the associated substance (which may therefore be referred to as -L-S_{c}).

A covalent linkage binds the reactive group R^{x}, the conjugated substance S_{c} or the ionic group R^{±} to the dye molecule, either directly via a single covalent bond which is depicted in the text as —R^{x}, —R^{±}, —S_{c}, or with a combination of stable chemical bonds (-L-), that include single, double, triple or aromatic carbon-carbon bonds; carbon-sulfur bonds, carbon-nitrogen bonds, phosphorus-sulfur bonds, nitrogen-nitrogen bonds, nitrogen-oxygen or nitrogen-platinum bonds, or aromatic or heteroaromatic bonds; -L- includes ether, thioether, carboxamide, sulfonamide, urea, urethane or hydrazine moieties. Preferably, -L- includes a combination of single carbon-carbon bonds and carboxamide or thioether bonds.

Where the substance is associated noncovalently, the association may occur through various mechanisms, including incorporation of the compound or precursor into or onto a solid or semisolid matrix, such as a bead or a surface, or by nonspecific interactions, such as hydrogen bonding, ionic bonding, or hydrophobic interactions (such as Van der Waals forces). The associated carrier may be selected from the group consisting of polypeptides, polynucleotides, polysaccharides, beads, microplate well surfaces, metal surfaces, semiconductor and non-conducting surfaces, nanoparticles, and other solid surfaces.

The associated or conjugated substance may be associated with or conjugated to more than one reporter compound, which may be the same or different. Generally, methods for the preparation of dye-conjugates of biological substances are well-known in the art. See, for example, Haugland et al., MOLECULAR PROBES HANDBOOK OF FLUORESCENT PROBES AND RESEARCH CHEMICALS, Eighth Edition (1996), or G. T. Hermanson, Bioconjugate Techniques, Academic Press, London, (1996), which is hereby incorporated by reference. Typically, the association or conjugation of a chromophore or luminophore to a substance imparts the spectral properties of the chromophore or luminophore to that substance.

Useful substances for preparing conjugates according to the present disclosure include, but are not limited to, amino acids, peptides, proteins, phycobiliproteins, nucleosides, nucleotides, nucleic acids, carbohydrates, lipids, ion-chelators, biotin, pharmaceutical compounds, nonbiological polymers, cells, and cellular components. The substance to be conjugated may be protected on one or more functional groups in order to facilitate the conjugation, or to insure subsequent reactivity.

Where the substance is a peptide, the peptide may be a dipeptide or larger, and typically includes 5 to 36 amino acids. Where the conjugated substance is a polypeptide, it may be a protein that is an enzyme, an antibody, lectin, protein A, protein G, hormones, or a phycobiliprotein. The conjugated substance may be a polynucleotide or nucleic acid polymer, such as for example DNA oligonucleotides, RNA oligonucleotides (or hybrids thereof), or single-stranded, double-stranded, triple-stranded, or quadruple-stranded DNA, or single-stranded or double-stranded RNA.

Another class of carriers includes carbohydrates that are polysaccharides, such as dextran, heparin, glycogen, starch and cellulose.

Where the substance is an ion chelator, the resulting conjugate may be useful as an ion indicator (calcium, sodium, magnesium, zinc, potassium and other important metal ions) particularly where the optical properties of the reporter-conjugate are altered by binding a target ion. Preferred ion-complexing moieties include crown ethers (U.S. Pat. No. 5,405,957) and BAPTA chelators (U.S. Pat. No. 5,453,517), among others.

The associated or conjugated substance may be a member of a specific binding pair, and therefore useful as a probe for the complementary member of that specific binding pair, each specific binding pair member having an area on the surface or in a cavity which specifically binds to and is complementary with a particular spatial and polar organization of the other. The conjugate of a specific binding pair member may be useful for detecting and optionally quantifying the presence of the complementary specific binding pair member in a sample, by methods that are well known in the art.

Representative specific binding pairs may include ligands and receptors, and may include but are not limited to the following pairs: antigen-antibody, biotin-avidin, biotin-streptavidin, IgG-protein A, IgG-protein G, carbohydrate-lectin, enzyme-enzyme substrate; ion-ion-chelator, hormone-hormone receptor, protein-protein receptor, drug-drug receptor, DNA-antisense DNA, and RNA-antisense RNA.

Preferably, the associated or conjugated substance includes proteins, carbohydrates, nucleic acids, drugs, and nonbiological polymers such as plastics, metallic nanoparticles such as gold, silver and carbon nanostructures among others. Further carrier systems include cellular systems (animal cells, plant cells, bacteria). Reactive dyes can be used to label groups at the cell surface, in cell membranes, organelles, or the cytoplasm.

Polymethines and squaraines have promising photophysical properties as red and NIR fluorescent dyes, but the usefulness of in particular squaraine dyes is often discriminated due to the susceptibility to chemical attack of the squaric acid ring moiety by nucleophiles. Recently, it was shown that permanent encapsulation of a squaraine dye, as the thread component in a Leigh-type rotaxane, provides tremendous chemical and photochemical stabilization [E. Arunkumar, et al., J. Am. Chem. Soc., 127 (2005) 3288]. The encapsulating macrocycle not only increases the chemical and photochemical stability of the squaraine thread but also inhibits aggregation-induced quenching of fluorescence and broadening of its absorption spectrum in water.

Finally these compounds can be linked to small molecules such as amino acids, vitamins, drugs, haptens, toxins, and environmental pollutants, among others. Another important ligand is tyramine, where the conjugate is useful as a substrate for horseradish peroxidase. Additional embodiments are described in U.S. Patent Application Publication No. US 2002/0077487.

Synthesis and Characterization

The central precursor for the synthesis of bis-dyes is 2,3,3,6,7,7-hexamethyl-3,7-dihydropyrrolo[2,3-f]indole which was described in F. A. Mihaijlenko and A. N. Boguslavskaya, Khimiya Geterotsykl. Soed. (in Russian), 1971; (5), p. 614-617. Various quarternization reactions of this molecule are described in Example 1. The incorporation is of the carboxy-pentyl residue and sulfo-butyl and/or sulfo-propyl residues into the 1,5 position of this molecule have not been described previously.

The synthesis of the disclosed reporter compounds typically is achieved in a multi-step reaction, starting with the synthesis of a methylene base and the dihydropyrrolo[2,3-f]indole. Typical starting materials include e.g. benzindoles, benzoselenzoles, benzoxazoles, benzimidazoles, squaric acid. etc. These starting materials may contain additional spacer groups in position 3 of the indolenine ring. The introduction of spacer groups and/or increasing the number of sulfonate groups may help to reduce the tendency of the dyes to aggregate in aqueous solution and when covalently bound to proteins.

The synthesis of cyanine dyes is described in Mujumdar et al., Bioconjugate Chem. 4(2) 105-111, 1993 and in several other patent applications (U.S. Patent Appl. US 2002/0077487 A1, U.S. Pat. No. 5,569,587, U.S. Pat. No. 5,672,027, U.S. Pat. No. 5,808,044). Bis-cyanine dyes of this disclosure exhibit absorption maxima in the range between 500 and 950 nm. In addition to other structural parameters, the selection of a monomethine, trimethine, or pentamethine linkage permits the spectral properties of the resulting compound to be altered according to the characteristics desired. In cyanines, where the remainder of the compound is held constant, shifting from a monomethine to a trimethine, to a pentamethine linkage in a W¹ or W² substituent typically results in a shift of the absorption and emission wavelengths of the resulting compounds to progressively longer wavelengths.

As compared to the monomeric versions, the absorption spectra of the bis-cyanine dyes of this disclosure can be red-shifted by about 100 nm. While the absorption of tri-cyanines (at typical example would be Cy3™) are around 550 nm, the absorption of bis-tricyanines (Example 5, compound 8) is shifted to around 650 nm and consequently the absorption of bis-pentamethine cyanines including squaraines can be found around 750 nm (Example 3, compound 5b). Upon substitution of the squaraine ring with dicayno-methylene group an additional shift of the absorption and emission maxima was obtained (Example 3, compound 5a).

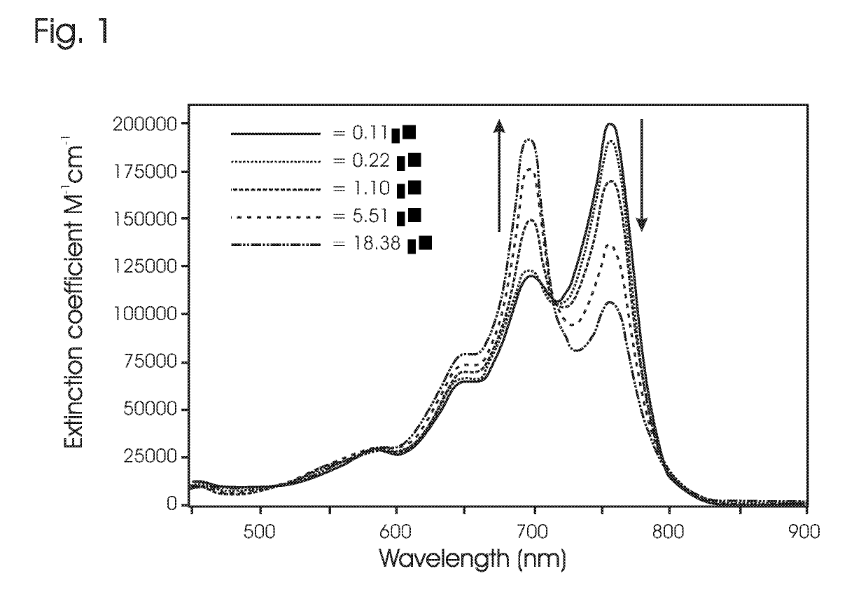

The emission of compound 5b is completely quenched in water but shows a weak emission band in methanol. The absorption of the bis-squaraine dye 5b in aqueous solutions (FIG. 1) shows two bands with a strong concentration dependence in the range between 0.2-20 μM. At concentrations below 1 μM, the longer wavelength band is the dominant band. With increasing concentrations the intensity of the 757 nm band decreases while intensity of the absorption band at 698 nm increases. The extinction coefficient is independent of the concentration. Upon covalent binding of dye 5b to BSA the 698 nm band is dominant at any given D/P ratio. Importantly, dye-conjugates of 5b are also non-fluorescent which makes these compound an ideal candidate as non-fluorescent acceptors for energy-transfer assays and applications. Its broad absorption spectrum from 600-800 nm makes it a promising fluorescence quencher for labels such as Cy5, Cy5.5, Alexa 647, Alexa 680 and Cy7.

Another non-fluorescent bis-cyanine derivative is compound 15 which has an absorption maximum around 550 nm. The compound is non-fluorescent in MeOH, EtOH and water and is perfectly suited as acceptor for cyanines (Cy3, Cy3.5) and xanthene-based dyes (Alexa 546, Alexa 555, Alexa 568, Rhodamine B) with emission in the is 500-600 nm range.

Asymmetrical dyes can be synthesized by reacting the mono-substituted, reactive versions of pyrrolo-indoles such as 13 and 14 with methylene bases with non-identical substitution. In this way mono-reactive bis-dyes can be synthesized.

To enhance water-solubility, sulfonic acid or other groups such as including quaternary ammonium, polyether, carboxyl, and phosphate, among others, may be introduced into the heterocyclic ring systems. In order to facilitate covalent attachment to proteins, reactive N-hydroxy-succinimide ester (NHS ester) or other forms may be synthesized

The absorption maxima can be fine-tuned by additional introduction of functional groups to match the emission lines of a frequency-doubled Nd-Yag laser (532 nm), Kr-ion laser (568 and 647 nm), the HeNe laser (543 nm and 633 nm) and diode lasers (635 nm, 650 nm, 780 nm etc.). Cyanine dyes exhibit a lesser tendency to change their quantum yields upon changing the environment (e.g. labelling to a protein).

Many compounds of the present disclosure possess an overall electronic charge. It is to be understood that when such electronic charges are present, that they are balanced by an appropriate counterion, which may or may not be identified.

### EXAMPLE 1

### Synthesis of Precursors

This section describes the synthesis of various precursors. p-hydrazinobenzene sulfonic acid (Illy et al., J. Org. Chem. 33, 4283-4285, 1968), 1-(5-carboxypentyl)-2,3,3-trimethyl-3H-5-indoliumsulfonate (1a), 2,3,3-trimethylindole-5-sulfonic acid potassium salt (1b), 1-(3-sulfonato propyl)-2,3,3-trimethylindoleninium-5-sulfonate (1h) (Mujumdar et al., Bioconj. Chem. 4(2) 105-111, 1993), and 1,2,3,3-tetramethylindoleninium-5-sulfonate (1c) were synthesized using literature procedures. 1d-1f are synthesized according to the procedures provided in U.S. Patent Application Publication No. 2002/0077487. 1-(2-phosphonethyl)-2,3,3-trimethylindoleninium-5-sulfonate (1i) is described in PCT Patent Application Publication No. WO 01/36973.

Other starting materials such p-hydranzino-phenylacetic acid and the relevant indolenine are described in Southwick et al., Cytometry 11, 418-430 (1990). Finally, starting materials for cationic dyes containing quaternary ammonium residues (trimethyl or triethyl ammonium) can be synthesized according to Hamilton et al. U.S. Pat. No. 6,140,494.

The synthesis of 7-(carboxypentyl)-2,3,3-trimethyl-3H-pyrrolo[2,3-b]pyridine and 5-bromo-7-(3-sulfopropyl)-2,3,3-trimethyl-3H-pyrrolo[2,3-b]pyrimidinium starting materials for the synthesis of the relevant squaraine dyes is described in U.S. Patent Application No. 2002/0077487.

1,3-Dithiosquaric acid disodium salt (2c) and triethylammonium 2-butoxy-3-dicyanomethylene-4-oxo-1-cyclobuten-1-olate (2d) were synthesized according to Seitz et al., Chem. Ber. 112, 990-999, (1979) and Gerecht et al., Chem. Ber. 117, 2714-2729 (1984), respectively.

The 3-cyanoimino-4-oxo-1-cyclobutene-1,2-diolate (2e) is synthesized starting from dibutylsquarate according to the procedure of K. Köhler et al. Chem. Ber. 118, 1903-1916 (1985). Disodium-3,4-dioxo-1-cyclobutene-1,2-dithiolate trihydrate 2f is synthesized according to R. West, JOC 41(24), 3904 (1976) or G. Seitz et al., Chem. Ber. 112, 990-999 (1979).

Relevant starting materials and compounds are also described A.-M. Osman et al., in Ind. J. Chem. 16B, October 1978, 865-868.

### Synthesis of 1-(5-carboxypentyl)-2,3,3-trimethyl-3H-5-indoliumsulfonate (1a) p-Hydrazinobenzenesulfonic acid

33 g of sodium carbonate was added to a suspension of 104 g (0.6 mol) of p-aminobenzenesulfonic acid in 400 mL of hot water. The solution was cooled to 5° C. in an ice-bath, and 70 g of concentrated sulfuric acid were added slowly under rapid stirring. A solution of 42 g of sodium nitrite in 100 mL of water was then added under cooling. A light yellow diazo-compound precipitate formed, which was filtered and washed with water, but not dried.

The wet diazo-compound was added under stirring and cooling (5° C.) to a solution of 170 g of sodium sulfite in 500 mL of water. The solution, which turned orange, was stirred under cooling for 1 h, and then heated to reflux. Finally, 400 mL of concentrated hydrochloric acid were added. The solution turned yellow, and the product precipitated as a white solid. For complete decoloration, 1-2 g of powdered zinc were added. The reaction mixture was cooled overnight, and the precipitate was filtered, washed with water, and dried in an oven at 100° C.

Yield: 96 g (85%), white powder; M.P.=286° C. (Lit.=285° C.); R_{f}: 0.95 (RP-18, water:MeOH 2:1).

### Synthesis of potassium 2,3,3-trimethylindoleninium-5-sulfonate (1b)

18.2 g (0.12 mol) of p-hydrazinobenzenesulfonic acid and 14.8 g (0.17 mol) of isopropylmethylketone were stirred in 100 mL of glacial acetic acid at room temperature for 1 h. The mixture was then refluxed for 4 h. The mixture was cooled to room temperature, and the resulting pink solid precipitate was filtered and washed with ether.

The precipitate was dissolved in methanol, and a concentrated solution of potassium hydroxide in 2-propanol was added until a yellow solid completely precipitated. The precipitate was filtered, washed with ether, and dried in a desiccator over P₂O₅.

Yield: 20.4 g (71%), off-white powder; M.P.=275° C.; R_{f}: 0.40 (silica gel, isopropanol:water:ammonia 9:0.5:1).

### 1-(5-carboxypentyl)-2,3,3-trimethyl-3H-5-indoliumsulfonate (1a)

15.9 g (57 mmol) of potassium 2,3,3-trimethylindoleninium-5-sulfonate and 12.9 g (66 mmol) of 6-bromohexanoic acid were refluxed in 100 mL of 1,2-dichlorobenzene for 15 min under a nitrogen atmosphere. The solution was cooled to room temperature, and the resulting pink precipitate was filtered, washed with chloroform, and dried.

Yield: 15.8 g (58%), pink powder; R_{f}: 0.75 (RP-18, MeOH:water 2:1).

### Synthesis of 1,2,3,3-tetramethylindolium-5-sulfonate (1c)

1.1 g of 2,3,3-trimethylindoleninium-5-sulfonate were suspended in 30 mL of methyl iodide. The reaction mixture was heated to boiling for 25 h in a sealed tube. After the mixture was cooled, excess methyl iodide was decanted, and the residue was suspended in 50 mL of acetone. The solution was filtered, and the residue was dried in a desiccator over CaCl₂. The resulting light yellow powder was used without further purification.

Yield: 90%, light yellow powder.

### Synthesis of 3-(5-carboxypentyl)-2,3-dimethyl-5-sulfo-1-(3-sulfopropyl) indolium sodium salt (1d), (Scheme I)

### Diethyl 3-acetyl-3-methylnonanedioate (IIa)

A mixture of 1.34 g (12 mmol) potassium t-butoxide and 10 g t-butanol was stirred and heated until the t-butoxide had been dissolved. The solution was cooled to about 50° C. and 1.7 g (11.8 mmol) of ethyl 2-methylacetoacetate (I) was added dropwise, Ethyl-6-bromohexanoate (3 g, 13.5 mmol) was then added dropwise and the reaction mixture was stirred and refluxed for 5 hours. The mixture was filtered and the solvent was removed under reduced pressure. The residue was partitioned between 1 M HCl and chloroform. The organic layer was dried over magnesium sulfate and purified on silica gel using 1:10 ethyl acetate/hexane as the eluent to yield 2.5 g (75%) of ethyl 2-(5-carboethoxypentyl)-2-methylacetoacetate (IIa) as yellow liquid.

### 7-methyl-8-oxononanoic acid IIIa

The above compound IIa (8.7 mmol) was dissolved in 30 ml of methanol. A solution of 1.05 g NaOH (26.3 mmol) in 15 mL water was added. The mixture was stirred and heated at 50° C. for 20 hours. The solution was reduced to about 10 mL, acidified to pH 1 and extracted with ethyl acetate. The organic phase was collected, dried over MgSO₄ and evaporated to yield 1.47 g (91%) of 7-methyl-8-oxononanoic acid (IIIa) as pale orange liquid.

### 6-(1,2-Dimethyl-6-sulfo-1H-1-indenyl)hexanoic acid (IVa)

The nonanoic acid IIIa (7.9 mmol) was refluxed in 15 mL of acetic acid with 1.46 g of 4-hydrazinobenzenesulfonic acid (7.75 mmol) for 5 hours. The acetic acid was evaporated and the product was purified on silica gel (RP-18, H₂O) to yield 1.45 g (55%) of the orange solid (IVa).

### Indolenine 1d

To the methanol solution of 1.1 g of Compound IVa is added 0.34 g of anhydrous sodium acetate. The mixture is stirred for five minutes. The solvent is evaporated and the resulting sodium salt is heated with 2.4 g of propane sultone at 110° C. for 1 hour to generate the final product 1d.

### Synthesis of 3-(6-hydroxyhexyl)-2,3-dimethyl-5-sulfo-1-(3-sulfopropyl) indolium, sodium salt (1e)

Starting material 1e is synthesized analogously to 1d using ethyl 2-methylacetoacetate and 6-benzoyl-1-bromo-hexane in presence of 1.2 equivalents of sodium hydride in THF. After isolating the 3-(6-hydroxyhexyl)-2,3-dimethyl-5-sulfo-indolium, inner salt the hydroxy group is again protected and the compound is quarternized using propanesultone. Deprotection is achieved using dilute NaOH.

1f is synthesized analogously taking into account the more polar nature of the sulfonic groups that are introduced either by reaction with 2-bromo-ethane-sulfonic acid, propane- or butanesultone. Sulfogroups can also be introduced by reaction of a 3-carboxy-alkyl-substituted compound like 1d with taurine according to Terpetschnig et al. Anal. Biochem. 217, 197-204 (1994).

Phosphate groups can be introduced in a similar way reacting ethyl 2-methylacetoacetate (I) with bromo-alkyl-phosphonates such as diethyl(3-bromopropyl)phosphonate or diethyl(2-bromoethyl)phosphonate (Aldrich) according to the above procedure (Scheme I). Conversion of the diethylphosphonates into the free acid is achieved by heating the compound in 47% HBr solution at reflux for 1.5-2 h.

Ionic and reactive groups may further be introduced into the indolenine by reacting a phenyl-hydrazine derivative with 2-acetyl-diethylmalonate or the relevant 2-acetyl-methylenetetraethyldiphosphonate as described in Organikum, pp 480-481, Deutscher Verlag der Wissenschaften, Berlin 1990, and subsequent cleavage of the esters as described above.

5-carboxy-derivatized indoles such as 1g that contain a spacer group in position 3 can be synthesized using 4-hydrazino-benzoic acid as described in Anal. Biochem. 217, 197-204 (1994) or 4-hydrazino-phenyl-acetic acid as described in Cytometry 11(3), 418-30 (1990), and reacting them in a Fisher indole synthesis with 7-methyl-8-oxononanonic acid or one of the other functionalized precursors as described above.

Other indolenine based starting materials that contain functional groups in R₃ and R₄ can be synthesized according to 1d using unsubstituted ethyl acetoacetate and 2.2 equivalents of the substituted halogen compound (ethyl-6-bromohexanoate, diethyl-3-bromopropyl-phosphonate, 6-benzoyl-1-bromo-hexane) and 2 equivalents of the potassium t-butoxide and are used as starting materials to synthesize the dyes of this disclosure. R₃ and R₄ can also be a part of an aliphatic ring system as described in U.S. Patent Application Publication No. 2002/0077487. 1j is synthesized analogously to compound 1a from the commercially available 2,3,3 trimethyl-indole and bromo-hexanoic acid.

Selected precursors are shown below

| | |
|---|---|
| | 1 |
| 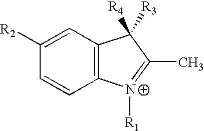 | |

| | | | R₃ | |
|---|---|---|---|---|
| 1 | R₁ | R₂ | (x = 2,3,4) R₄ | |
| a | (CH₂)₅COOH | SO₃⁻ | CH₃ | CH₃ |
| b | — | SO₃K | CH₃ | CH₃ |
| c | CH₃ | SO₃⁻ | CH₃ | CH₃ |
| d | (CH₂)₃SO₃Na | SO₃⁻ | (CH₂)₅COOH | CH₃ |
| e | (CH₂)₃SO₃Na | SO₃⁻ | (CH₂)₆OH | CH₃ |
| f | (CH₂)₃SO₃Na | SO₃⁻ | (CH₂)ₓSO₃Na | CH₃ |
| g | (CH₂)₃SO₃⁻ | COOH | (CH₂)₅COOH | CH₃ |
| h | (CH₂)₃SO₃Na | SO₃⁻ | CH₃ | CH₃ |
| i | (CH₂)₂PO(OH)₂ | SO₃⁻ | CH₃ | CH₃ |
| j | (CH₂)₅COOH | H | CH₃ | CH₃ |

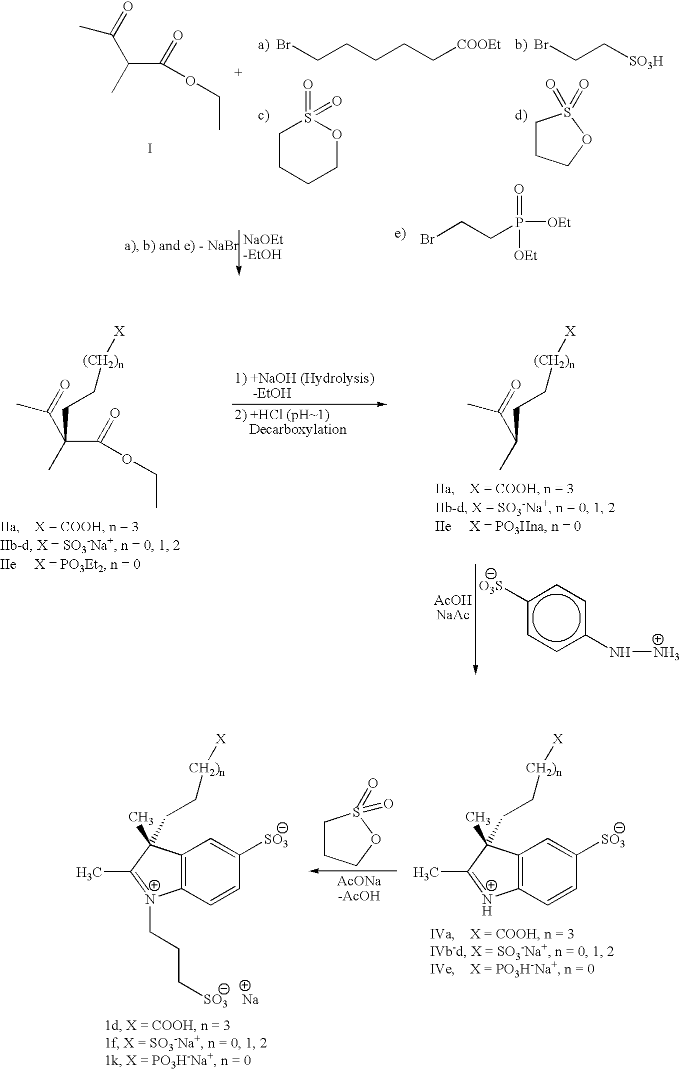

| | |
|---|---|
| | 2 |
| 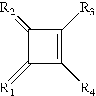 | |

| 2 | R₁ | R₂ | R₃ | R₄ |
|---|---|---|---|---|
| a | O | O | OH | OH |
| b | O | O | OC₄H₉ | OC₄H₉ |
| c | S | O | S⁺ Na⁺ | O⁻ Na⁺ |
| d | O | C(CN)₂ | OC₄H₉ | O⁻ HNEt₃⁺ |
| e | N—CN | O | O⁻ K⁺ | O⁻ K⁺ |
| f | O | O | S⁻ Na⁺ | S⁻ Na⁺ |

Synthesis of 2,3,3,6,7,7-hexamethyl-3,7-dihydropyrrolo[2,3-f]indole (3) according to F. A. Mihaijlenko and A. N. Boguslavskaya, Khimiya Geterotsykl. Soed. (in Russian), 1971; (5), p. 614-617.

3-[4-(1,1-dimethyl-2-oxopropylamino)anilino]-3-methyl-2-butanone was synthesized according to F. A. Mihaijlenko and A. N. Boguslavskaya, Khimiya Geterotsykl. Soed. (in Russian), 1971; (5), p. 614-617.

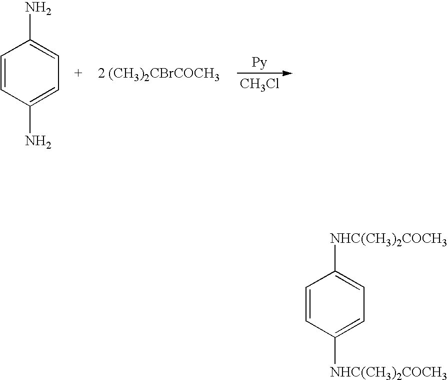

3 g (0.028 mol) of p-phenylenediamine was dissolved in 50 ml of chloroform at heating and 9.6 g (0.12 mol) of pyridine was added. Then the solvent of 10 g (0.06 mol) of bromoketone in 15 ml of chloroform was added dropwise under stirring. The mixture was refluxed for 3 h. The solvent was removed under reduced pressure by a rotary evaporator. Viscous brawn residue was treated by concentrated ammonia to give the precipitate, which was filtered off and washed with water to pH 7. The product was crystallized from chloroform. Yield: 1.7 g (22%). mp 175-178° C. Found: N, 10.05, requires N, 10.14%.].

δ_{H} (200 MHz, DMSO-d₆) 6.22 (4H, s, arom H), 5.29 (2H, s, NH), 2.08 (6H, s, COCH₃), 1.24 (12H, s, C(CH₃)₂).

### 2,3,3,6,7,7-hexamethyl-3,7-dihydropyrrolo[2,3-f]indole (3)

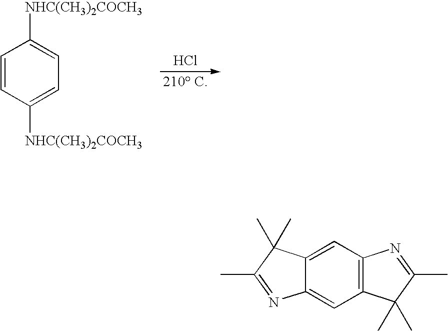

1.2 g of 3-[4-(1,1-dimethyl-2-oxopropylamino)anilino]-3-methyl-2-butanone was dissolved in 15 ml of concentrated hydrochloric acid and evaporated until dry. Then the residue was heated under argon at 210° C. for 45 min. After cooling the precipitate was dissolved in water and neutralized with ammonia to yield 0.7 g (67%) of the product 3. mp 190-195° C. δ_{H} (200 MHz, DMSO-d₆) 7.45 (2H, s, arom H), 2.18 (6H, s, CH₃), 1.24 (12H, s, C(CH₃)₂).

### 1,2,3,3,5,6,7,7-octamethyl-3,7-dihydropyrrolo[2,3-f]indolediium diiodide (3a)

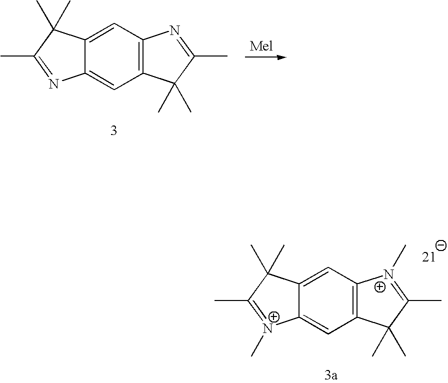

0.45 g of benzodipyrrolenyl 3 was refluxed in 5 ml of methyl iodide for 7 hours. The reaction mixture was diluted with ether, the precipitate was filtered, washed with ether and dissolved in chloroform. The residue 3a was filtered and washed with chloroform. Yield 0.45 g. Found: N, 5.74, requires N, 5.30%.

### 1,2,3,3,5,6,7,7-octamethyl-3,7-dihydropyrrolo[2,3-f]indolediium di(4-methyl-1-benzenesulfonate) (3b)

A mixture of 3 g of 2,3,3,6,7,7-hexamethyl-3,7-dihydropyrrolo[2,3-f]indole 3 and 9.3 g of methyl 4-methyl-1-benzenesulfonate was melted for 4 hours at 140-145° C., treated with acetone, filtered, washed with acetone, and dried. Yield: 7.1 g (92%). δ_{H} (200 MHz, DMSO-d₆) 8.51 (2H, s, arom H), 7.47 (4H, d, 7.6 Hz, Ts arom H), 7.11, (4H, d, 7.6 Hz, Ts arom H), 4.0 (6H, s, N⁺CH₃), 2.8 (6H, s, 2-CH₃), 2.28 (6H, s, Ts CH₃), 1.57 (12H, s, 3-CH₃).

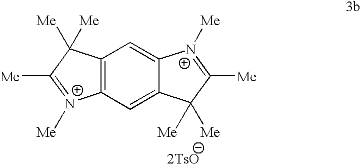

### 1,5-diethyl-2,3,3,6,7,7-hexamethyl-3,7-dihydropyrrolo[2,3-f]indolediium di(4-methyl-1-benzenesulfonate) (3c)

A mixture of 0.5 g of 2,3,3,6,7,7-hexamethyl-3,7-dihydropyrrolo[2,3-f]indole 3 and 1.25 g of ethyl 4-methyl-1-benzenesulfonate were heated for 4 h at 150-155° C., treated with acetone, filtered, washed with acetone, and dried. Yield: 830 mg (62%). δ_{H} (200 MHz, DMSO-d₆) 8.6 (2H, s, arom H), 7.47 (4H, d, 8.2 Hz, Ts arom H), 7.11 (4H, d, 8.2 Hz, Ts arom H), 4.62-4.37 (4H, m, N⁺CH₂CH₃), 2.88 (6H, s, 2-CH₃), 2.28 (6H, s, Ts CH₃), 1.59 (12H, s, indolenine CH₃), 1.52-1.38 (6H, m, N⁺CH₂CH₃).

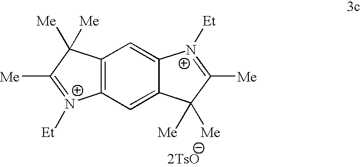

### 1,5-di(5-carboxypentyl)-2,3,3,6,7,7-hexamethyl-3,7-dihydropyrrolo[2,3-f]indolediium dibromide (3d)

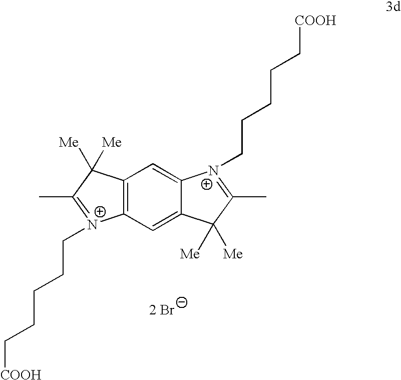

A mixture of 200 mg of 2,3,3,6,7,7-hexamethyl-3,7-dihydropyrrolo[2,3-f]indole 3 and 1250 mg of 6-bromohexanoic acid were heated for 3 h at 130-140° C., treated with hot isopropanol, filtered, washed with acetone, and dried. Yield: 370 mg (71%).

### 4-[2,3,3,6,7,7-hexamethyl-5-(4-sulfonatobutyl)-3,7-dihydropyrrolo[2,3-f]indolediium-1-yl]-1-propanesulfonate (3e)

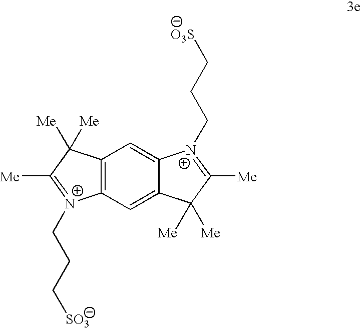

A mixture of 0.2 g of 2,3,3,6,7,7-hexamethyl-3,7-dihydropyrrolo[2,3-f]indole 3 and 0.56 g of propane sultone were heated at 130-140° C. for 20 h, treated with boiling isopropanol, filtered, washed with acetone, and purified by column chromatography (RP-18, water) Yield: 0.3 g

δ_{H} (200 MHz, DMSO-d₆) 8.7 (2H, s, ArH), 4.69 (4H, m, N⁺—CH₂—), 2.87 (6H, s, CH₃), 2.64 (4H, m, —CH₂—), 2.16 (4H, m, S—CH₂—), 1.59 (12H, s, CH₃);

FAB-MS (NBA) m/z 486 (M2H)⁺.

### 1,3,3,5,7,7-hexamethyl-2,6-dimethylene-1,2,3,5,6,7-hexahydropyrrolo[2,3-f]indole (3f)

1.048 g of (1.7 mmol) 1,2,3,3,5,6,7,7-octamethyl-3,7-dihydropyrrolo[2,3-f]indoledinium di(4-methyl-1-benzenesulfonate) 3b was dissolved in minimal volume of water and 1 ml of aqueous ammonia (25%) was added (pH≈9), heated to 80° C. for 1 h and the product 3f was filtered off. Yield: 450 mg (98%).

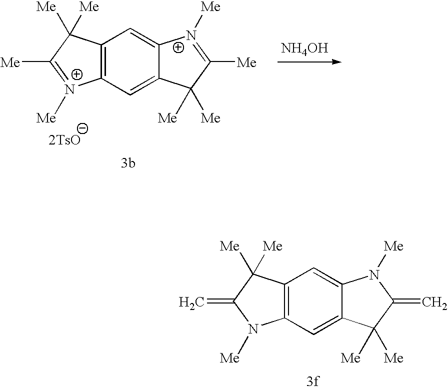

δ_{H} (200 MHz, DMSO-d₆) 6.62 (2H, s, bispyrrolenin ArH), 3.72 (4H, d, J=3.3 Hz, CH₂), 2.96 (6H, s, N—CH₃), 1.27 (12H, s, CH₃).

### 2-[1,3,3,5,7,7-hexamethyl-6-(2-oxoethylidene)-5,7-dihydropyrrolo[2,3-f]indol-2(1H,3H)-ylidene]acetaldehyde (3g)

0.21 ml of POCl₃ was added to 2 ml of dry DMF dropwise at 10° C. The mixture was stirring for 1 hour and 286 mg of 1,3,3,5,7,7-hexamethyl-2,6-dimethylene-1,2,3,5,6,7-hexahydropyrrolo[2,3-f]indole 3f in 2 ml of DMF was added. A mixture refluxed for 1 h, cooled and poured into a solution of 2.5 g of NaOH in 15 ml water, and stirred for 30 min at room temperature. Product 3g was filtered off and washed several times with water. Yield: 280 mg (86%).

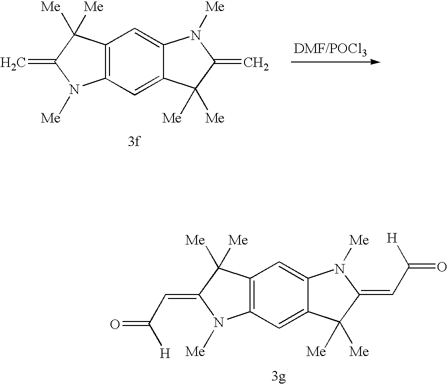

δ_{H} (200 MHz, DMSO-d₆) 9.84 (2H, d, J=8.8 Hz, —CO—H), 7.31 (2H, s, bispyrrolenin ArH), 5.24 (2H, d, J=8.8 Hz, CH) 3.26 (6H, s, N—CH₃), 1.61 (12H, s, CH₃);

FAB-MS (NBA) m/z 324 (M)⁺, 325 (MH)⁺.

### EXAMPLE 2

### Synthesis of symmetrical bis-squarylium dyes (5)

### Triethylammonium 3-dicyanomethylene-4-oxo-2-(1,3,3-trimethyl-2,3-dihydro-1H-2-indolylidenmethyl)-1-cyclobuten-1-olate (4)

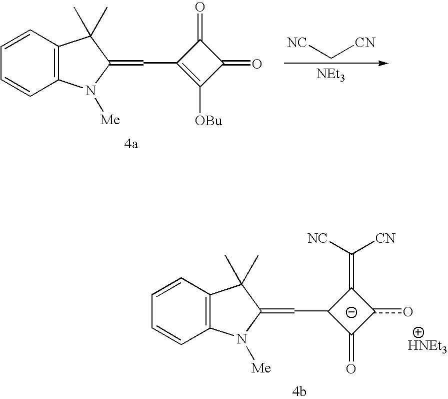

1 ml (7.14 mmol) of TEA was added dropwise to a mixture of 2 g (6.15 mmol) of mono-substituted squaraine 4a (A. Tartarets et al., Dyes & Pigments, 64, 125-134, 2005), 440 mg (6.66 mmol) of malononitrile in 35 ml of ethanol and stirred for 2 h at room temperature. The solvent was removed under reduced pressure. The raw product was column purified (Silica gel 60, 0-2% methanol-chloroform) to give (2.52 g, 98%) 4b as orange crystals, mp 153° C.; Analysis: N, 13.44 C₂₅H₃₀N₄O₂ requires N, 13.39%; δ_{H} (200 MHz, DMSO-d₆) 8.74 (1H, br s, NH⁺), 7.29 (1H, d, 7.5 Hz, arom H), 7.20 (1H, t, 7.5 Hz, arom H), 6.95 (1H, d, 8.3 Hz, arom H), 6.93 (1H, t, 7.8 Hz, arom H), 5.92 (1H, s, CH), 3.25 (3H, s, NCH₃), 3.11 (6H, q, 7.3, 14.6 Hz, N(CH₂CH₃)₃), 1.59 (6H, s, C(CH₃)₂), 1.20 (9H, t, 7.3 Hz, N(CH₂CH₃)₃); FAB-MS (glycerol) m/z 419 (MH⁺); IR (KBr) 2232 (CN), 2208 (CN), 1744 (CO), 1684, 1652 cm⁻¹.

### Hydrophobic symmetrical dye 5a

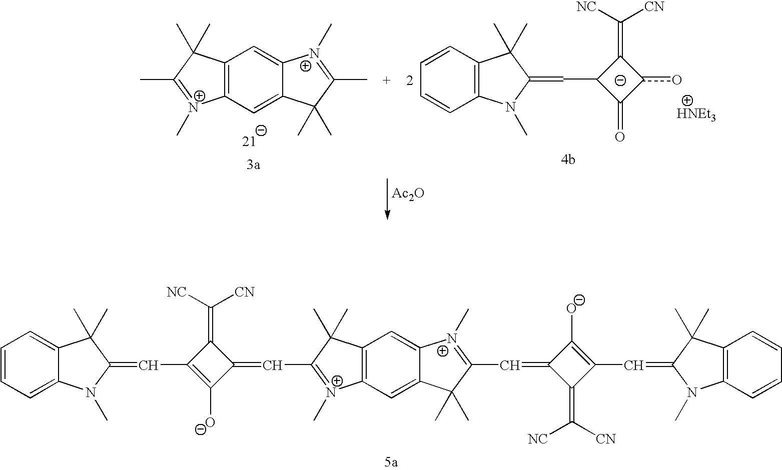

360 mg (0.86 mmol) 4b and 210 mg (0.40 mmol) 1,2,3,3,5,6,7,7-octamethyl-3,7-dihydropyrrolo[2,3-f]indoledinium-di(4-methyl-1-benzenesulfonate) 3a were heated under reflux in 30 ml of acetic anhydride for 5 h. The solvent was removed under reduced pressure by a rotary evaporator. The residue was purified by a column chromatography (Silica gel 60, chloroform) to give product 5a (90 mg, 26%); UV: λₘₐₓ (abs) 810 nm (CHCl₃), λₘₐₓ (fl) 824 nm (CHCl₃); δ_{H} (200 MHz, DMSO-d₆) 7.55 (2H, s, arom H), 7.50 (2H, d, 7.5 Hz, arom H), 7.40-7.28 (4H, m, arom H), 7.25-7.16 (2H, m, arom H), 6.30 (2H, s, CH), 6.19 (2H, s, CH), 3.64 (6H, s, NCH₃), 3.52 (6H, s, NCH₃), 1.68 (12H, s, indolenine CH₃), 1.66 (12H, s, indolenine CH₃);

### Water-soluble, reactive, symmetrical bis-squarylium dye (5b)

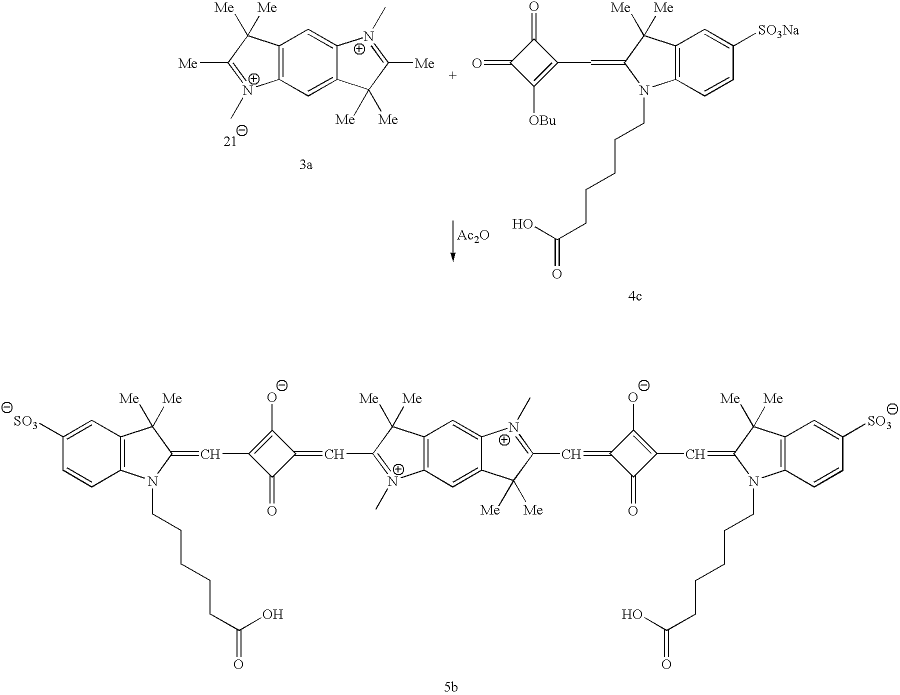

240 mg (0.46 mmol) 4c (B. Oswald, et al. Bioconjugate Chem. 10, 925-931, 1999; Terpetschnig E. A., et al. U.S. Pat. No. 6,538,129, 2003) and 120 mg (0.23 mmol) 3b were heated under reflux in 10 ml of acetic anhydride for 2 h. The solvent was removed under reduced pressure by a rotary evaporator. The residue was purified by a column chromatography (PR-18, MeOH/H₂O) to give product 5b (80 mg, 35%); UV: λₘₐₓ (abs) 756 nm (water), λₘₐₓ (abs) 764 nm (EtOH), λₘₐₓ (fl) 780 nm (EtOH); ε=200.000 (water); δ_{H} (200 MHz, DMSO-d₆) 7.73-7.17 (8H, arom H), 5.79 (4H, s, CH), 4.16-3.95 (4H, m, NCH₂), 3.66 (3H, s, NCH₃), 2.28-2.14 (4H, m, CH₂COO), 1.734 (12H, s, CH₃), 1.68 (12H, s, CH₃), 1.6-1.27 (12H, m, —CH₂—);

### EXAMPLE 3

### Synthesis of 2,6-di[(1E,3E)-4-(4-dimethylaminophenyl)-1,3-butadienyl]-1,3,3,5,7,7-hexamethyl-3,7-dihydropyrrolo[2,3-f]indolediium di(4-methyl-1-benzenesulfonate) (6)

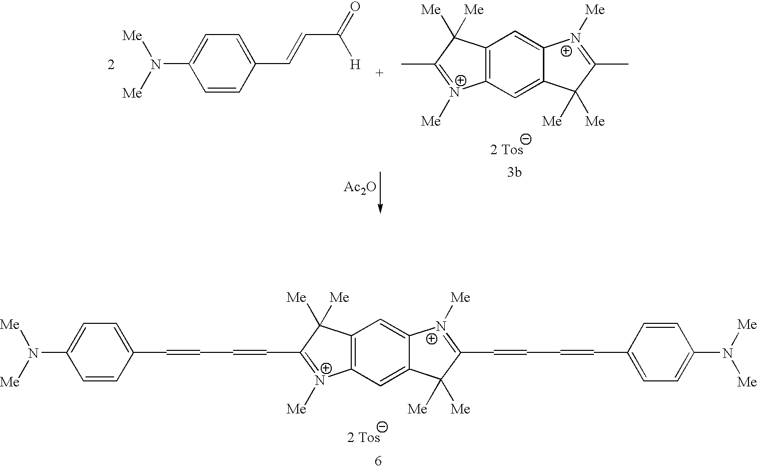

150 mg (0.86 mmol) of 3-(4-dimethylaminophenyl)acrylaldehyde was dissolved in 5 ml of acetic anhydride, and 209 mg (0.34 mmol) of 1,2,3,3,5,6,7,7-octamethyl-3,7-dihydropyrrolo[2,3-f]indoledinium di-4-methyl-1-benzenesulfonate 3b was added. The mixture was refluxed for 1 hour. After cooling the solvent was removed under reduced pressure. The residue was treated with hexane, filtered and washed with hexane and Et₂O. The remaining solid was redissolved in a minimum volume of nitromethane and precipitated with Et₂O. Yield 250 mg 6 (80%). UV: λₘₐₓ (abs) 727 nm (MeOH) λₘₐₓ (abs) 766 nm (CHCl₃), λₘₐₓ (fl) 810 nm (CHCl₃). Very weak fluorescence in CHCl₃ and no fluorescence in MeOH. δ_{H} (200 MHz, DMSO-d₆) 8.34 (2H, t, 14 Hz, CH), 8.24 (2H, s, bispyrrolenin arom. H), 7.78 (2H, d, 14 Hz, CH), 7.61 (4H, d, 7.7 Hz, arom H), 7.47 (4H, d, 7.6 Hz, Tos H), 7.32 (2H, t, 14 Hz, CH), 7.1 (4H, d, 7.6 Hz, Tos H), 6.93 (2H, d, 14 Hz, CH), 6.85 (4H, d, 7.7 Hz, arom H), 3.9 (6H, s, N⁺CH₃), 3.1 (12H, s, NCH₃), 2.28 (6H, s, Tos CH₃), 1.77 (12H, s, indolenine CH₃).

### EXAMPLE 4

1,3,3,5,7,7-hexamethyl-2,6-di[3-(1,1,3-trimethyl-2,3-dihydro-1H-2-indenyliden)-1-propenyl]-3,7-dihydropyrrolo[2,3-f]indolediium-di(4-methyl-1-benzenesulfonate) (7) was synthesized according to (Mihajlenko F. A, Boguslavskaya A. N, Kiprianov A. I.; Khimiya Geterotsykl. Soed. (in Russ), 1971; No 5, p. 618-620).

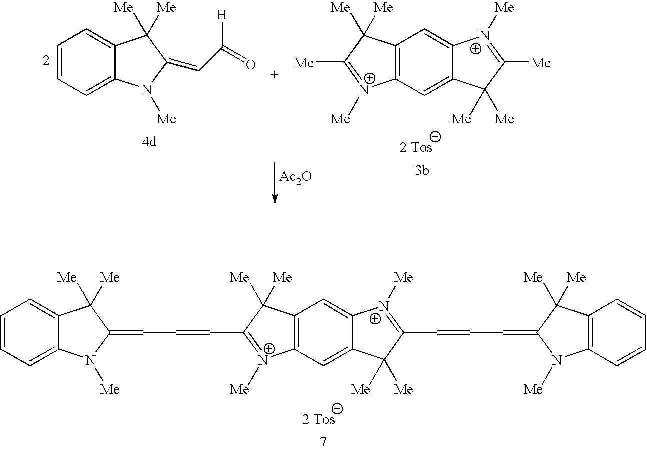

150 m g (0.75 mmol) of 2-(1,1,3-trimethyl-2,3-dihydro-1H-2-indenyliden) acetaldehyde 4d (H. Fritz, Chemische Berichte; 1959, 92 (8), 1809-17); was dissolved in 5 ml of acetic anhydride, and 182 mg (0.34 mmol) of 1,2,3,3,5,6,7,7-octamethyl-3,7-dihydropyrrolo[2,3-f]indoledinium di(4-methyl-1-benzenesulfonate) 3b was added. The mixture was refluxed for 1 hour. After cooling the solvent was removed under reduced pressure by a rotary evaporator. The residue was treated by hexane, filtered off and washed with hexane and Et₂O. Solid was redissolved in a minimum volume of nitrometan and precipitated with Et2O. Yield 180 mg (62%). UV: λₘₐₓ (abs) 658 nm (CHCl₃), λₘₐₓ (abs) 644 nm (MeOH), λₘₐₓ (abs) 645 nm (6 mg/ml BSA), λₘₐₓ (fl) 677 nm (CHCl₃), λₘₐₓ (fl) 664 nm (MeOH), λₘₐₓ (fl) 668 nm (6 mg/ml BSA); Q.Y. 10.4%; Q.Y._{BSA} 6.6%; δ_{H} (200 MHz, DMSO-d₆) 8.31 (2H, t, 13.4 Hz, CH), 7.85 (2H, s, bispyrrolenin arom H), 7.65 (2H, d, arom H), 7.53-7.39 (4H, Tos and 4H indolenine H), 7.38-7.25 (2H, m, arom indolenine H), 7.11 (4H, d, 7.8 Hz, Tos H), 6.45 (4H, d, 13.4 CH), 3.7 (6H, s, bispyrrolenin NCH₃), 3.66 (6H, s, indolenine NCH₃), 2.28 (6H, s, Tos CH₃), 1.73 (12H, s, bispyrrolenin CH₃) 1.7 (12H, s, indolenine CH₃).

FAB-MS (GI) m/z 621 (Cat−CH₃)⁺, 635 (Cat−H)⁺, 636 (Cat⁺⁺+e⁻)⁺, 807 (Cat+An)⁺.

### EXAMPLE 5

Symmetrical bis-cyanine dye (8) Intermediate (9) was synthesized according to (Mihajlenko F. A., Dyadyusha G. G., Boguslavskaya A. N.; Khimiya Geterotsykl. Soed. (in Russ.), 1975, No 3, 370-376).

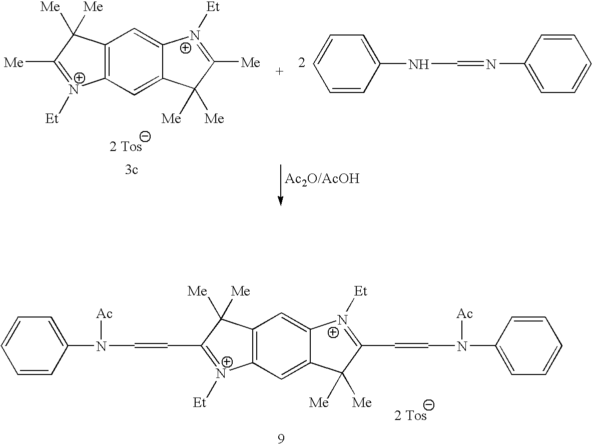

210 mg of 1,5-diethyl-2,3,3,6,7,7-hexamethyl-3,7-dihydropyrrolo[2,3-f]indoledinium di(4-methyl-1-benzenesulfonate) 3c are dissolved in a mixture of 10 ml of acetic anhydride and 5 ml of acetic acid, and 160 mg of diphenylformamidine were added. The solution was boiled under reflux for 1 hour. After cooling the solvent was removed under reduced pressure and residue was treated with hexane, filtered and washed with Et₂O and acetone and dried. Yield 160 mg (53%).

### Symmetrical bis-cyanine dye (8)

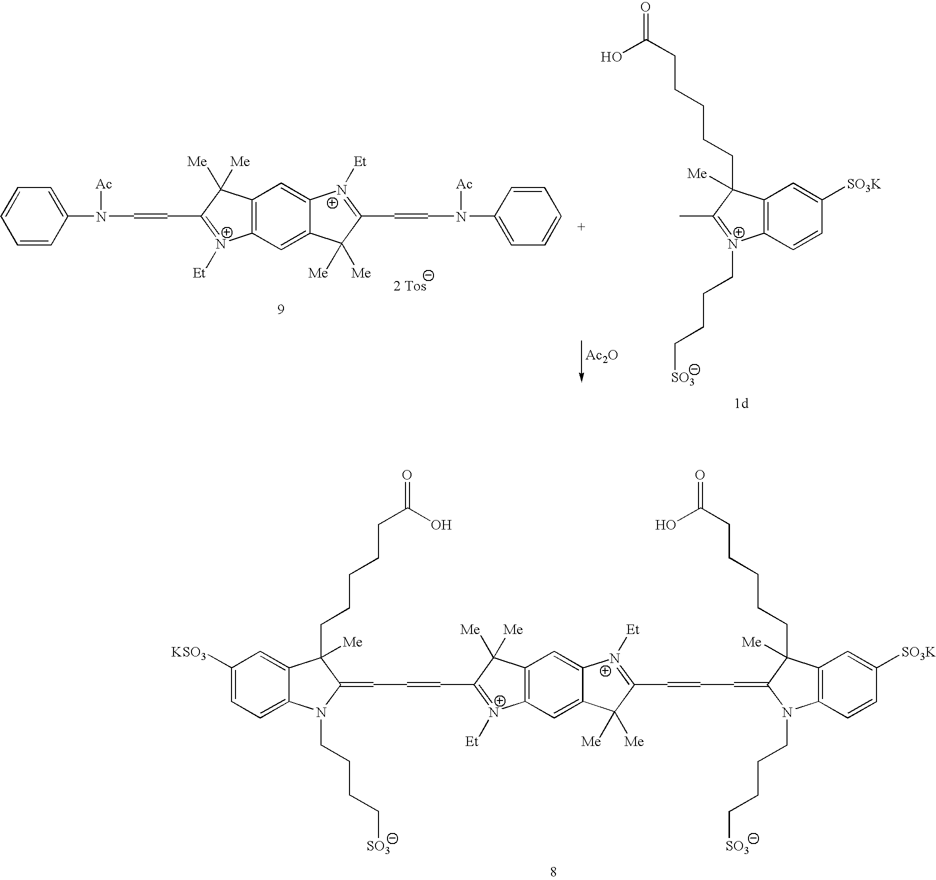

160 mg (0.172 mmol) of intermediate 9 were dissolved in a mixture of 3 ml of acetic anhydride and 3 ml of pyridine, and 300 mg (0.543 mmol) of potassium 3-(5-carboxypentyl)-2,3-dimethyl-1-(4-sulfonatobutyl)-3H-5-indoliumsulfonate 1d were added. The solution was heated under reflux for 1 hour, cooled to RT and the product was precipitated with ether and filtered. The solids was washed with ether and dried. The raw product was column purified (PR-18, MeOH/H₂O) to give (30 mg, 10%) 8. UV: λₘₐₓ (abs) 654 nm (water), λₘₐₓ (fl) 670 nm (water), Q.Y. 5%, ε 157,000. δ_{H} (200 MHz, DMSO-d₆) 8.30 (2H, t, 13.3 Hz, β CH), 7.88 (2H, s, arom H), 7.76 (2H, s, arom H), 7.68 (2H, d, 8.4 Hz, arom H), 7.46 (2H, d, 8.4 Hz, arom H), 4.42-3.98 (8H, m, NCH₂), 2.23-1.94 (8H, m, CH₂SO₃H), 1.93-0.40 (18H, m, indolenine CH₃, bis-pyrrolenin CH₃ and other aliphatic CH₂).

### EXAMPLE 6

### 2,6-di[4-(3,5-diphenyl-4,5-dihydro-1H-1-pyrazolyl)styryl]-1,3,3,5,7,7-hexamethyl-3,7-dihydropyrrolo[2,3-f]indolediium diiodide (10)

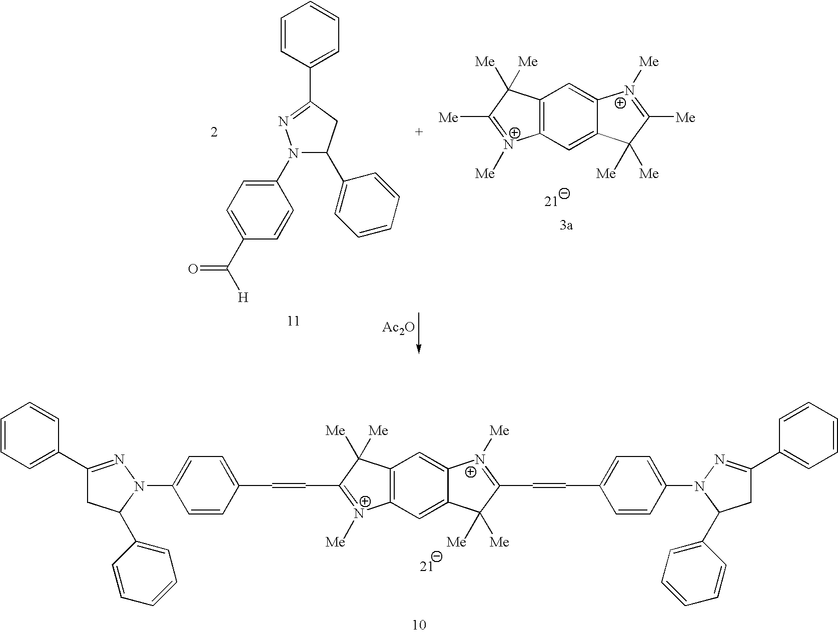

140 mg (0.429 mmol) of 4-(3,5-diphenyl-4,5-dihydro-1H-1-pyrazolyl)benz-aldehyde 11 [L. A. Kutulya, A. E. Shevchenko, Yu.N.Surov; Khimiya Geterotsykl. Soed. (in Russian), 1975, No. 2, 250-253] were dissolved in 5 mL of acetic anhydride, 100 mg (0.191 mmol) of 1,2,3,3,5,6,7,7-octamethyl-3,7-dihydropyrrolo[2,3-f]indoledinium diiodide 3a were added and the mixture was refluxed for 30 min. After cooling, the solvent was removed under reduced pressure. The residue was treated with hexane, filtered and washed with hexane and ether. The solid was redissolved in a minimum volume of nitromethane and precipitated with ether. Yield: 120 mg 10 (57%). UV: λₘₐₓ (abs) 697 nm (CHCl₃), 677 nm (MeOH), λₘₐₓ (fl) 740 nm (CHCl₃), λₘₐₓ (fl) 735 nm (MeOH).

### EXAMPLE 7

### 2,6-di(4-dimethylaminostyryl)-1,3,3,5,7,7-hexamethyl-3,7-dihydropyrrolo[2,3-f]indolediium diiodide (12)

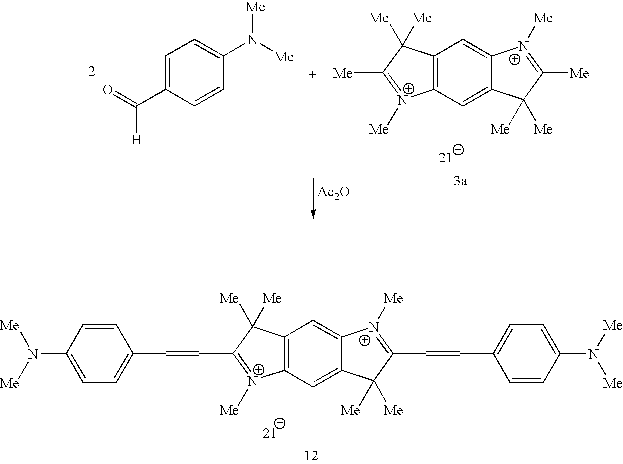

60 mg (0.403 mmol) of 4-dimethylaminobenzaldehyde was dissolved in 5 ml of acetic anhydride, and 100 mg (0.191 mmol) of 1,2,3,3,5,6,7,7-octamethyl-3,7-dihydropyrrolo[2,3-f]indoledinium diiodide 3a was added. The mixture was refluxed for 40 min. After cooling the solvent was removed under reduced pressure by a rotary evaporator. The residue was treated by hexane, filtered off and washed with hexane and Et₂O. Solid was redissolved in a minimum volume of nitromethane and precipitated with Et₂O. Yield 90 mg 12 (60%). UV: λₘₐₓ (abs) 649 nm (EtOH), 643 nm (MeOH), 650 nm (CHCl₃), λₘₐₓ (fl) 687 nm (EtOH), λₘₐₓ (fl) 682 nm (MeOH), λₘₐₓ (fl) 683 nm (CHCl₃); δ_{H} (200 MHz, DMSO-d₆) 8.34 (2H, d, 15.6 Hz, CH), 8.22 (2H, s, bispyrrolenin arom H), 8.10 (4H, d, 8.5 Hz, arom H), 7.25 (2H, d, 15.6 Hz, CH), 6.93 (4H, d, 8.5 Hz, arom H), 4.01 (6H, s, N⁺—CH₃), 3.18 (12H, s, N(CH₃)₂), 1.81 (12H, s, 13.4 bispyrrolenin CH₃).

### EXAMPLE 8

### 1-(5-carboxypentyl)-2-((Z)-1-3-[(E)-1-(1,5-diethyl-3,3,6,7,7-pentamethyl-3,7-dihydro pyrrolo[2,3-f]indolediium-2-yl)methylidene]-2-olato-4-oxo-1-cyclobutenylmethylidene)-3,3-dimethyl-5-indolinesulfonate (13)

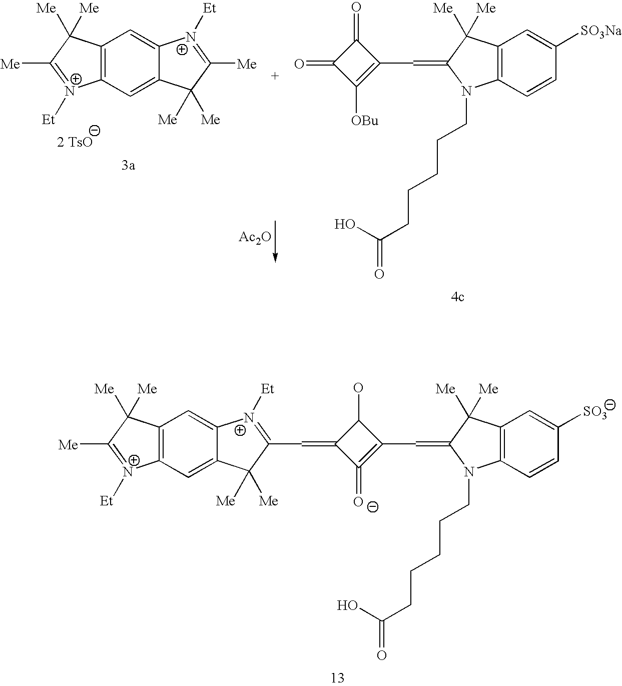

210 mg (0.33 mmol) of 1,5-diethyl-2,3,3,6,7,7-hexamethyl-3,7-dihydropyrrolo[2,3-f]indoledinium di(4-methyl-1-benzenesulfonate) 3c, 150 mg (0.28 mmol) of sodium 2-[(Z)-1-(2-butoxy-3,4-dioxo-1-cyclobutenyl)methylidene]-1-(5-carboxypentyl)-3,3-dimethyl-5-indolinesulfonate 4c and 7 ml of acetic anhydride were refluxed for 10 hours. The product was precipitated with benzene. The solids were washed with benzene and CHCl₃ and dried. After drying the product was dissolved in MeOH, filtered and the solvent removed under reduced pressure. The residue was purified by column chromatography (PR-18, EtOH/water) to give product 13 (45 mg, 25%);

### EXAMPLE 9

### Sodium 1-(5-carboxypentyl)-3,3-dimethyl-2-((Z)-1-2-olato-4-oxo-3-[(E)-1-(3,3,6,7,7-pentamethyl-3,7-dihydropyrrolo[2,3-t]indol-1-ium-2-yl)methylidene]-1-cyclobuten-ylmethylidene)-5-indolinesulfonate (14)

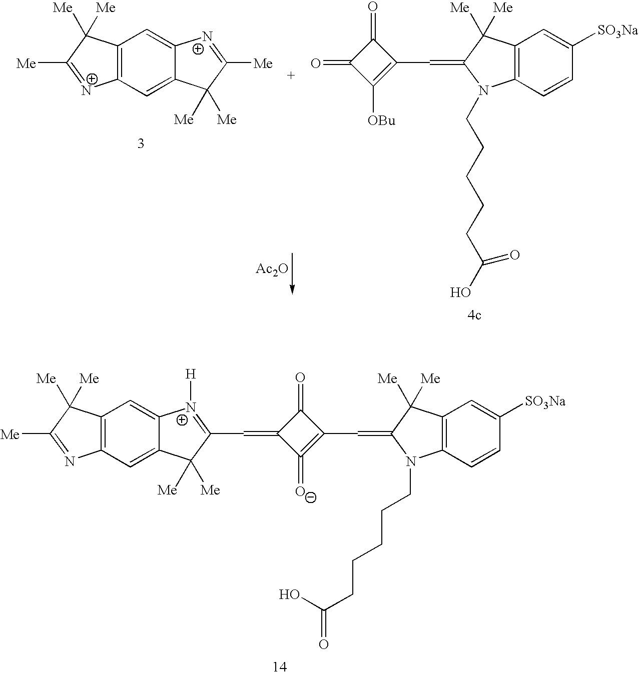 A mixture of 100 mg (0.4 mmol) of 2,3,3,6,7,7-hexamethyl-3,7-dihydropyrrolo[2,3-f]indole 3, 200 mg (0.35 mmol) of sodium 2-(2-butoxy-3,4-dioxo-1-cyclobutenylmethylene)-1-(5-carboxypentyl)-3,3-dimethyl-5-indolinesulfonate 4c, and 7 ml of acetic anhydride was refluxed for 48 hours. After precipitation with benzene the oiled product was washed with benzene and dried. The product was dissolved in ethanol, filtered, and the solvent was removed under reduced pressure. The residue was purified by column chromatography (PR-18, EtOH/water, gradient) to give product 14 (40 mg, 20%); UV: λₘₐₓ (abs) 648 nm (H₂O), λₘₐₓ (abs) 667 nm (6 mg/ml BSA), λₘₐₓ (fl) 663 nm (H₂O), λₘₐₓ (fl) 666 nm (MeOH), λₘₐₓ (fl) 675 nm (BSA); ε174.000 (water), Q.Y. 4% (water), Q.Y. 4% (MeOH), Q.Y. 12% (6 mg/ml BSA); δ_{H} (200 MHz, DMSO-d₆) 13.56 (1H, s, N⁺H), 7.76-6.76 (5H, m, arom H), 5.65 (2H, s, —CH═), 4.05 (2H, m, N⁺CH₂), 2.3-2.13 (5H, m, 2-CH₃ and CH₂COOH), 1.78-1.19 (12H, m, 2-CH₃ and 3-CH₃ of bis-pyrrolenine, 3-CH₃ of indolenine and α,β,γ aliphatic CH₂).

### EXAMPLE 10

### Synthesis of 6-(2-(1,3,3,5,7,7-hexamethyl-6-(2-(1,3,3-trimethyl-1,2,3,4-tetrahydro benzo[h]quinazolin-3-ium-6-yl)vinyl)-3,7-dihydropyrrolo[2,3-f]indolediium-2-yl)vinyl)-1,3,3-trimethyl-1,2,3,4-tetrahydrobenzo[h]quinazolin-3-ium di(4-methyl-1-benzene sulfonat) di(hexafluorophosphate) (15)

6-formyl-1,3,3-trimethyl-1,2,3,4-tetrahydrobenzo[h]quinazolin-3-ium hexafluorphosphate (16) was synthesized according to O. N. Semenova, Yu.A.Kudryavtseva, I. G. Ermolenko, and L. D. Patsenker. Behavior of Dimethylamino-naphthalenes in the Vilsmeier-Haak Reaction. Russian Journal of Organic Chemistry, 2005, V. 41, No. 7, p. 1100].

A mixture of 1.7 g (10 mmol) of 1-dimethylaminonaphthalene in 4.2 mL of DMF was heated to 40° C. Then 3.7 mL (40 mmol) of POCl₃ were added dropwise and heated at 80° C. for 15 min. After that the reaction mixture was poured into ice, neutralized with sodium acetate, 10 mmol of NH₄ PF₆ were added, and solid of 16 was filtered off. Yield: 82%. M.P. 223-225° C. 6H (200 MHz, DMSO-d₆) 10.25 (1H, s, COH), 9.23 (5H, d, 8.3 Hz, H⁵), 8.21 (1H, d, 8.3 Hz, H⁸), 7.90 (1H, s, H³), 7.63-7.84 (2H, m, H⁶ and H⁷), 4.99 (2H, s, CH₂), 4.84 (2H, s, CH₂), 3.56 (3H, s, NCH₃), 3.20 (6H, s, N⁺(CH₃)₂). Analysis: N, 6.32%. Requires N, 6.17%.

### 6-(2-(1,3,3,5,7,7-hexamethyl-6-(2-(1,3,3-trimethyl-1,2,3,4-tetrahydrobenzo[h]quinazolin-3-ium-6-yl)vinyl)-3,7-dihydropyrrolo[2,3-f]indolediium-2-yl)vinyl)-1,3,3-tri methyl-1,2,3,4-tetrahydrobenzo[h]quinazolin-3-ium di(4-methyl-1-benzene sulfonat) di(hexafluorophosphate) (15)

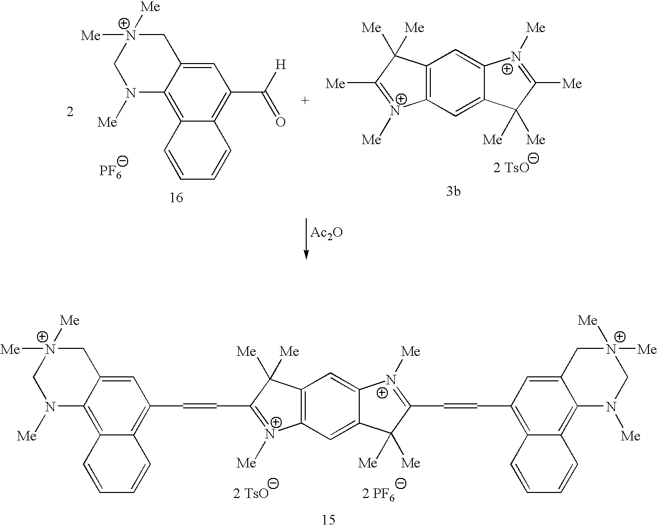

100 mg (0.25 mmol) of 6-formyl-1,3,3-trimethyl-1,2,3,4-tetrahydrobenzo[h]quinazolin-3-ium hexafluorophosphate 16 was dissolved in 5 ml of acetic anhydride, and 77 mg (0.125 mmol) of 1,2,3,3,5,6,7,7-octamethyl-3,7-dihydropyrrolo[2,3-f]indoledinium di(4-methyl-1-benzene sulfonate) 3b was added. The mixture was refluxed for 1 hour. After cooling the solvent was removed under reduced pressure and the residue treated with hexane, filtered and washed with hexane and Et₂O. The solid was redissolved in a minimum volume of nitromethane and precipitated with Et₂O. Yield: 95 mg 15 (55%). UV: λₘₐₓ (abs) 554 nm (MeOH).

### EXAMPLE 11

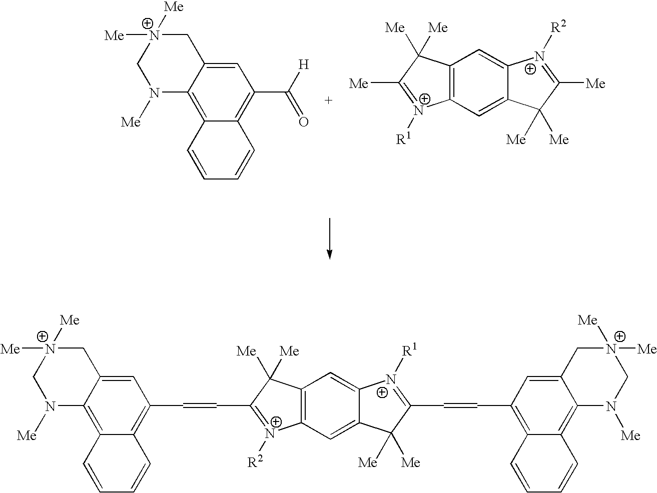

R¹ and R² are (CH₂)ₙCOOR(R═H, NHS-ester), (CH₂)ₙSO₃H (n=1-4)

### EXAMPLE 12

### 2-{3-[1,3,3,5,7,7-hexamethyl-6-[3-(1-methyl-2-quinoliniumyl)-2-propenylidene]-5,7-dihydropyrrolo[2,3-f]indol-2(1H,3H)-ylidene]-1-propenyl}-1-methyl quinolinium diiodide (17)

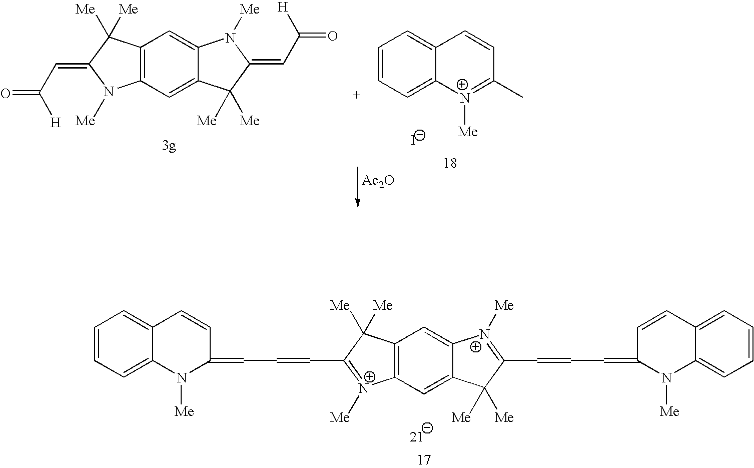

50 mg (0.154 mmol) of 2-[1,3,3,5,7,7-hexamethyl-6-(2-oxoethylidene)-5,7-dihydropyrrolo[2,3-f]indol-2(1H,3H)-ylidene]acetaldehyde 3g were dissolved in 2 ml of acetic anhydride, and 105 mg (0.368 mmol) of 1,2-dimethylquinolinium iodide 18 (L. F. Tietze, T. Eicher. Reaktionen und Synthesen im organish-chemischen Praktikum und Forschungslaboratorium. Georg Thieme Verlag Stuttgart New York, 1991) were added. The mixture was refluxed for 1 hour. After cooling, the solid was filtered, washed with ether. The raw product 17 was redissolved in a minimum volume of nitromethane and precipitated with ether. Yield: 88 mg.

UV: λₘₐₓ (abs) 692 nm (CHCl₃), λₘₐₓ (abs) 666 nm (MeOH), λₘₐₓ (fl) 734 nm (CHCl₃), λₘₐₓ (fl) 731 nm (MeOH).

δ_{H} (200 MHz, DMSO-d₆) 8.43-7.55 (14H, m, ArH, β methyn CH), 6.72 (2H, d, J=13.0 Hz, CH), 6.28 (2H, d, J=13.0 Hz, CH) 4.11 (6H, s, N—CH₃), 3.58 (6H, s, N—CH₃), 1.73 (12H, s, CH₃).

FAB-MS (GI) m/z 589 (Cat−CH₃)⁺, 603 (Cat−H)⁺.

### EXAMPLE 13

### 2-{3-[1,3,3,5,7,7-hexamethyl-6-[3-(1-methyl-2-pyridiniumyl)-2-propenylidene]-5,7-dihydropyrrolo[2,3-f]indol-2(1H,3H)-ylidene]-1-propenyl}-1-methylpyridinium di(4-methylbenzenesulfonate) (19)

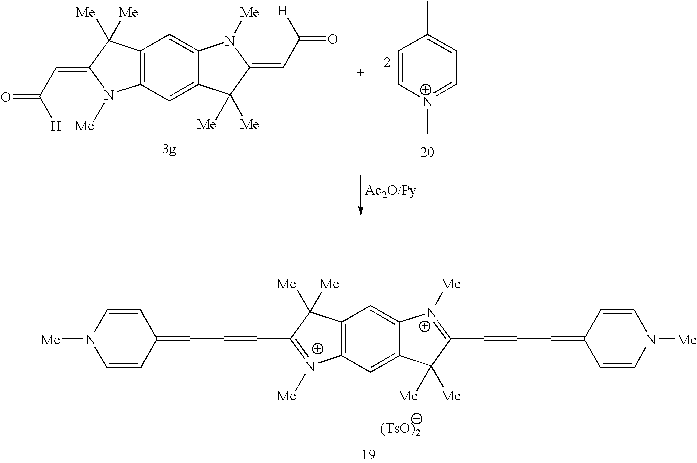

25 mg (0.077 mmol) of 2-[1,3,3,5,7,7-hexamethyl-6-(2-oxoethylidene)-5,7-dihydropyrrolo[2,3-f]indol-2(1H,3H)-ylidene]acetaldehyde 3g were dissolved in mixture of 2 ml of acetic anhydride and 1 ml of pyridine, and 61 mg (0.218 mmol) of 1,4-dimethylpyridinium 4-methylbenzenesulfonate 20 (L. F. Tietze, T. Eicher. Reaktionen und Synthesen im organish-chemischen Praktikum und Forschungslaboratorium. Georg Thieme Verlag Stuttgart New York, 1991) were added. The mixture was heated for 14 hour. After cooling, the solid was filtered, washed with ether. The raw product 19 was redissolved in a minimum volume of nitromethane and precipitated with ether. Yield: 40 mg (26%).

### EXAMPLE 14

### 2-{3-[1,3,3,5,7,7-hexamethyl-6-[3-(3-methyl-1,3-benzothiazol-3-ium-2-yl)-2-propen ylidene]-5,7-dihydropyrrolo[2,3-f]indol-2(1H,3H)-ylidene]-1-propenyl}-3-methyl-1,3-benzothiazol-3-ium diiodide (21)

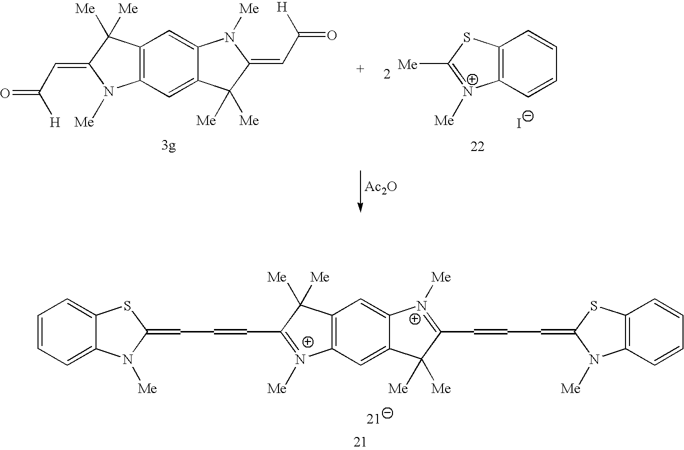

19 mg (0.059 mmol) of 2-[1,3,3,5,7,7-hexamethyl-6-(2-oxoethylidene)-5,7-dihydropyrrolo[2,3-f]indol-2(1H,3H)-ylidene]acetaldehyde 3g were dissolved in 2 ml of acetic anhydride, and 41 mg (0.141 mmol) of 2,3-dimethyl-1,3-benzothiazol-3-ium iodide 22 (Mills, W. H., JACS, 1922, 121, 455) were added. The mixture was refluxed for 1 hour. After cooling, the solid was filtered, washed with ether and crystallized from nitromethane. Yield: 35 mg 21 (68%).

UV: λₘₐₓ (abs) 659 nm (CHCl₃), λₘₐₓ (abs) 641 nm (MeOH), λₘₐₓ (fl) 687 nm (CHCl₃), λₘₐₓ (fl) 674 nm (MeOH).

δ_{H} (200 MHz, DMSO-d₆) 8.08 (2H, d, J=8.2 Hz, ArH), 7.98 (2H, t, 13.5 Hz, CH), 7.87 (2H, d, J=8.2, ArH), 7.73 (2H, s, bispyrrolenin ArH), 7.65 (2H, t, J=8.2, ArH), 7.51 (2H, t, J=8.2 Hz, ArH), 6.76 (2H, d, J=113.5 Hz, CH), 6.25 (2H, d, J=13.5 Hz, CH), 3.93 (6H, s, N—CH₃), 3.61 (6H, s, N—CH₃), 1.69 (12H, s, bispyrrolenin CH₃).

FAB-MS (GI) m/z 601 (Cat−CH₃)⁺, 615 (Cat−H)⁺, 584 (Cat⁺⁺+e⁻)⁺.

### 4-(3-{1,3,3,5,7,7-hexamethyl-6-[3-(1-methyl-4-quinoliniumyl)-2-propenylidene]-1,2,3,5,6,7-hexahydropyrrolo[2,3-f]indol-2-yliden}-1-propenyl)-1-methylquinolinium di(4-methyl-1-benzenesulfonate) (23)

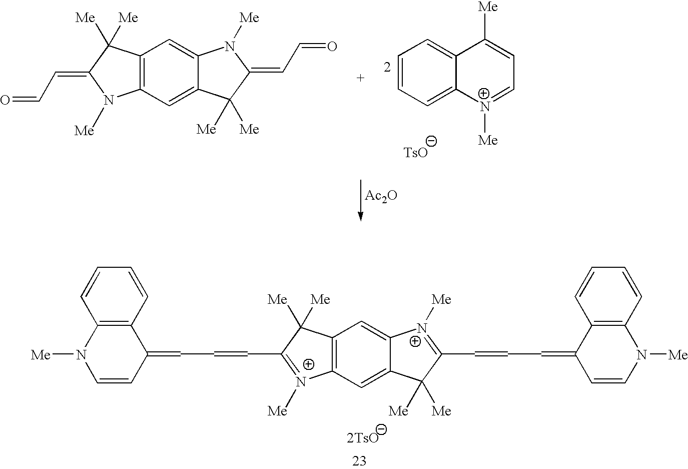

90 mg (0.105 mmol) of 1,4-dimethylquinolinium 4-methyl-1-benzenesulfonate were dissolved in 3 ml of acetic anhydride and 34 mg (0.052 mmol) of 2-[1,3,3,5,7,7-hexamethyl-6-(2-oxoethylidene)-5,7-dihydropyrrolo[2,3-f]indol-2(1H,3H)-ylidene]acetaldehyde 3g were added. The mixture was refluxed for 40 min. After cooling, product was precipitated with ether. Solid was crystallized from 2-propanol. Yield 35 mg (35%).

UV: λₘₐₓ (abs) 707 nm (CHCl₃), λₘₐₓ (abs) 717 nm (MeOH), λₘₐₓ (fl) 755 nm (CHCl₃), λₘₐₓ (fl) 773 nm (MeOH).

δ_{H} (200 MHz, DMSO-d₆) 8.63-7.71 (12H, m, 6.7 Hz chinolin ArH), 8.31 (2H, t, 13.4 Hz, β-CH), 7.55 (2H, s, bispyrrolenin ArH), 7.48 (4H, d, Ts ArH), 7.10 (4H, d, 7.8 Hz, Ts arom H), 7.25 (2H, d, 13.4 Hz, α-CH), 6.23 (2H, d, 13.4 Hz, α-CH), 4.22 (6H, s, chinolin N—CH₃), 3.53 (6H, s, bispyrrolenin N—CH₃), 2.27 (6H, s, Ts CH₃), 1.72 (12H, s, bispyrrolenin CH₃).

FAB-MS (GI) m/z 589 (Cat−CH₃)⁺, 603 (Cat−H)⁺, 604 (Cat⁺⁺+e⁻)⁺.

### 1,3,3,5,7,7-hexamethyl-2,6-di[3-(3-methyl-2,3-dihydro-1,3-benzoxazol-2-yliden)-1-propenyl]-3,7-dihydropyrrolo[2,3-f]indolediiumdi(4-methyl-1-benzenesulfonate) (24)

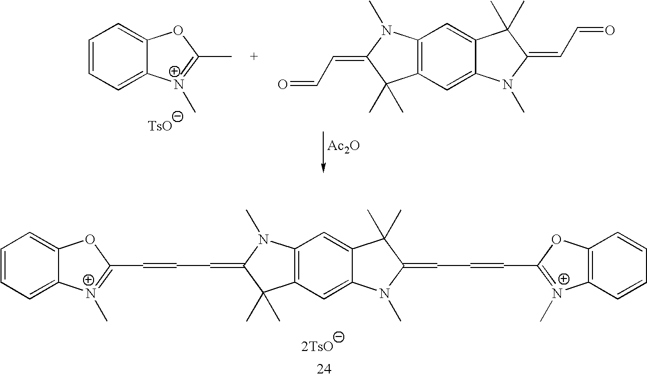 21 mg (0.065 mmol) of 2-[1,3,3,5,7,7-hexamethyl-6-(2-oxoethylidene)-5,7-dihydropyrrolo[2,3-f]indol-2(1H,3H)-ylidene]acetaldehyde 3g were dissolved in 2 ml of acetic anhydride, and 45 mg (0.141 mmol) of 2,3-dimethyl-1,3-benzoxazol-3-ium 4-methyl-1-benzenesulfonate were added. The mixture was refluxed for 40 min. After cooling, solids were filtered, washed with ether. Yield: 30 mg (50%) 24.

UV: λₘₐₓ (abs) 616 nm (CHCl₃), λₘₐₓ (abs) 596 nm (MeOH), λₘₐₓ (fl) 640 nm (CHCl₃), λₘₐₓ (fl) 629 nm (MeOH).

δ_{H} (200 MHz, DMSO-d₆) 8.29 (2H, t, J=13.5 Hz, CH), 7.86 (2H, d, 7.0 Hz, ArH), 7.77 (2H, d, J=7.0 Hz, ArH), 7.70 (2H, s, bispyrrolenin ArH), 7.60-7.49 (4H, m, ArH), 7.46 (4H, d, J=7.6 Hz, Ts ArH), 7.10 (4H, d, J=7.6 Hz, Ts ArH), 6.31 (2H, d, J=13.5 Hz, CH), 6.20 (2H, d, J=13.5 Hz, CH), 3.81 (6H, s, N—CH₃), 3.59 (6H, s, N—CH₃), 2.28 (6H, s, Ts CH₃), 1.69 (12H, s, bispyrrolenin CH₃).

FAB-MS (GI) m/z 569 (Cat−CH₃)⁺, 583 (Cat−H)⁺, 584 (Cat⁺⁺+e⁻)⁺, 755 (Cat+An)⁺.

### EXAMPLE 15

### General Protein Labelling Procedures and Determination of Dye-to-Protein Ratios

Protein labelling reactions were carried out using a 50 mM bicarbonate buffer (pH 9.1). A stock solution of 1 mg of dye in 100 μL of anhydrous DMF was prepared. 10 mg of protein were dissolved in 1 mL of 100 mM bicarbonate buffer (pH 9.1). Dye from the stock solution was added, and the mixture was stirred for 24 h at room temperature.

Unconjugated dye was separated from labelled proteins using gel permeation chromatography with SEPHADEX G50 (0.5 cm×20 cm column) and a 22 mM phosphate buffer solution (pH 7.3) as the eluent. The first colored band contained the dye-protein conjugate. A later blue band with a much higher retention time contained the separated free dye. A series of labelling reactions as described above were set up to obtain different dye-to-protein ratios. Compared to the free forms, the protein-bound forms of the dyes show distinct changes in their spectral properties.

Protein concentration was determined using the BCA Protein Assay Reagent Kit from Pierce (Rockford, Ill.). The dye-to-protein ratio (D/P) gives the number of dye molecules covalently bound to the protein.

Covalent Attachment of NHS-Esters to Polyclonal Anti-HSA

385 μL (5.2 mg/mL) of anti-HSA were dissolved in a 750 μL bicarbonate buffer (0.1 M, pH 9.0). 1 mg of NHS-ester is dissolved in 50 μL of DMF and slowly added to the above-prepared protein solution with stirring. After 20 h of stirring, the protein-conjugate was separated from the free dye using Sephadex G50 and a phosphate buffer (22 mM, pH 7.2). The first blue- or green-colored fraction that is isolated contains the labeled conjugate.

Conjugation of an NHS-Ester to BSA

0.5 mg of reactive dye in 50 μL of DMF was slowly added to a stirred solution of 5 mg of HSA in 750 μL of bicarbonate buffer (0.1 M, pH 9.0). The mixture was stirred for another 6 h at room temperature. The mixture was dialyzed against a phosphate buffer (22 mM, pH 7.2) using a dialysis membrane (1500 FT, Union Carbid) with a cutoff of 10.000.

Spectral Properties of Representative Dyes:

Spectral properties for various dyes of this disclosure were measured. The following table summarizes absorption (excitation) and emission spectral data for various dyes in organic solvents and in phosphate buffer (PB).

TABLE 1
Spectral propeties of selected dyes of the disclosure

| | | λₘₐₓ(abs) | λₘₐₓ(em) | ε | Q.Y. |
|---|---|---|---|---|---|
| Squaraine | Solvent | [nm] | [nm] | [L/(mol*cm)] | [%] |
| 5a | CHCl₃ | 810 | 824 | — | — |
| 5b | water | 756 | none | 200.000 | n.f. |
| 6 | MeOH | 727 | — | — | — |
| 7 | MeOH | 644 | 664 | — | 10.4 |
| 7-BSA | PB | 645 | 668 | — | 6.6 |
| 8 | water | 654 | 670 | 157.000 | 5 |
| 10 | CHCl₃ | 697 | 740 | — | — |
| 12 | MeOH | 643 | 682 | — | — |
| 14 | PB | 648 | 663 | 174.000 | 4 |
| 14-BSA | PB | 667 | 675 | — | 12 |
| 15 | MeOH | 554 | none | — | n.f. |

n.f. = not fluorescent

Description of Applications of Compositions of the Present Disclosure

The reporter compounds disclosed above exhibit utility for a variety of useful methods for various assay formats.

The assay may be a competitive assay that includes a recognition moiety, a binding partner, and an analyte. Binding partners and analytes may be selected from the group consisting of biomolecules, drugs, and polymers, among others. In some competitive assay formats, one or more components are labeled with photoluminescent compounds in accordance with the disclosure. For example, the binding partner may be labeled with such a photoluminescent compound, and the displacement of the compound from an immobilized recognition moiety may be detected by the appearance of fluorescence in a liquid phase of the assay. In other competitive assay formats, an immobilized enzyme may be used to from a complex with the fluorophore-conjugated substrate.

Some of these reporter molecules contain specific moieties for specific labelling of protein tyrosine phosphatases, as well as other phosphatases as described in Zhu, Q., et al.: Tetrahedron Letters, 44, 2669 (2003).

The binding of antagonists to a receptor can be assayed by a competitive binding method in so-called ligand/receptor assays. In such assays, a labeled antagonist competes with an unlabeled ligand for the receptor binding site. One of the binding partners can be, but not necessarily has to be, immobilized. Such assays may also be performed in microplates. Immobilization can be achieved via covalent attachment to the well wall or to the surface of beads.

Other preferred assay formats are immunological assays. There are several such assay formats, including competitive binding assays, in which labeled and unlabeled antigens compete for the binding sites on the surface of an antibody (binding material). Typically, there are incubation times required to provide sufficient time for equilibration. Such assays can be performed in a heterogeneous or homogeneous fashion.

Sandwich assays may use secondary antibodies and excess binding material may be removed from the analyte by a washing step.

Other types of reactions include binding between avidin and biotin, protein A and immunoglobulins, lectins and sugars (e.g., concanavalin A and glucose).

Certain dyes of the present disclosure are charged due to the presence of sulfonic groups. These compounds are impermeant to membranes of biological cells. In this case treatments such as electroporation and shock osmosis can be used to introduce the dye into the cell. Alternatively, such dyes can be physically inserted into the cells by pressure microinjection, scrape loading etc.

The reporter compounds described here also may be used to sequence nucleic acids and peptides. For example, fluorescently-labeled oligonucleotides may be used to trace DNA fragments. Other applications of labeled DNA primers include fluorescence in-situ hybridization methods (FISH) and for single nucleotide polymorphism (SNIPS) applications, among others.

Multicolor labeling experiments may permit different biochemical parameters to be monitored simultaneously. For this purpose, two or more reporter compounds are introduced into the biological system to report on different biochemical functions. The technique can be applied to fluorescence in-situ hybridization (FISH), DNA sequencing, fluorescence microscopy, and flow cytometry. One way to achieve multicolor analysis is to label biomolecules such as nucleotides, proteins or DNA primers with different luminescent reporters having distinct luminescence properties. Luminophores with narrow emission bandwidths are preferred for multicolor labeling, because they have only a small overlap with other dyes and hence increase the number of dyes possible in a multicolor experiment. Importantly, the emission maxima have to be well separated from each other to allow sufficient resolution of the signal. A suitable multicolor triplet of fluorophores would include a Cy3-analog of this disclosure, TRITC, and a Cy5-analog as described herein, among others.

Phosphoramidites are useful functionalities for the covalent attachment of dyes to oligos in automated oligonucleotide synthesizers. They are easily obtained by reacting the hydroxyalkyl-modified dyes of the present disclosure with 2-cyanoethyl-tetraisopropyl-phosphorodiamidite and 1-H tetrazole in methylene chloride.

The simultaneous use of FISH (fluorescence in-situ hybridization) probes in combination with different fluorophores is useful for the detection of chromosomal translocations, for gene mapping on chromosomes, and for tumor diagnosis, to name only a few applications. One way to achieve simultaneous detection of multiple sequences is to use combinatorial labeling. The second way is to label each nucleic acid probe with a luminophore with distinct spectral properties. Similar conjugates can be synthesized from the compounds of the disclosure and can be used in a multicolor multisequence analysis approach.

In another approach the dyes of the disclosure might be used to directly stain or label a sample so that the sample can be identified and/or quantitated. Such dyes might be added/labeled to a target analyte as a tracer. Such tracers could be used e.g. in photodynamic therapy where the labeled compound is irradiated with a light source and thus producing singlet oxygen that helps to destroy tumor cells and diseased tissue samples.

The reporter compounds of the present disclosure can also be used for screening assays for a combinatorial library of compounds. The compounds can be screened for a number of characteristics, including their specificity and avidity for a particular recognition moiety.

Assays for screening a library of compounds are well known. A screening assay is used to determine compounds that bind to a target molecule, and thereby create a signal change which is generated by a labeled ligand bound to the target molecule. Such assays allow screening of compounds that act as agonists or antagonists of a receptor, or that disrupt a protein-protein interaction. It also can be used to detect hybridization pr binding of DNA and/or RNA.

Other screening assays are based on compounds that affect the enzyme activity. For such purposes, quenched enzyme substrates of the present disclosure could be used to trace the interaction with the substrate. In this approach, the cleavage of the fluorescent substrate leads to a change in the spectral properties such as the excitation and emission maxima, intensity and/or lifetime, which allows to distinguish between the free and the bound luminophore.

The reporter compounds disclosed above may also be relevant to single molecule fluorescence microscopy (SMFM) where detection of single probe molecules depends on the availability of a fluorophore with high fluorescence yield, high photostability, and long excitation wavelength.

The dye compounds are also useful for use as biological stains. The dyes are not harmful and non-toxic to cells and other biological components. There may be limitations in some instances to the use of the above compounds as labels. For example, typically only a limited number of dyes may be attached to a biomolecules without altering the fluorescence properties of the dyes (e.g. quantum yields, lifetime, emission characteristics, etc.) and/or the biological activity of the bioconjugate. Typically quantum yields may be reduced at higher degrees of labeling. Encapsulation into beads offers a means to overcome the above limitation for the use of such compounds as fluorescent markers. Fluorescent beads and polymeric materials are becoming increasingly attractive as labels and materials for bioanalytical and sensing applications. Various companies offer particles with defined sizes ranging from nanometers to micrometers. Noncovalent encapsulation in beads may be achieved by swelling the polymer in an organic solvent, such as toluene or chloroform, containing the dye. Covalent encapsulation may be achieved using appropriate reactive functional groups on both the polymer and the dyes.

In general, hydrophobic versions of the disclosed compounds may be used for non-covalent encapsulation in polymers, and one or more dyes could be introduced at the same time. Surface-reactive fluorescent particles allow covalent attachment to molecules of biological interest, such as antigens, antibodies, receptors etc. Hydrophobic versions of the disclosed compounds, such as dyes having lipophilic substituents such as phospholipids, may non-covalently associate with lipids, liposomes, lipoproteins. They may also be useful for probing membrane structure and membrane potentials.

Compounds of the present disclosure may also be attached to the surface of metallic nanoparticles such as gold or silver nanoparticles or metal-coated surfaces. It has recently been demonstrated that fluorescent molecules may show increased quantum yields near metallic nanostructures e.g. gold or silver nanoparticles (O. Kulakovich et al. Nanoletters 2 (12) 1449-52, 2002, E. Matveeva et al., Anal. Biochem. 363 (2007) 239-245). This enhanced fluorescence may be attributable to the presence of a locally enhanced electromagnetic field around metal nanostructures. The changes in the photophysical properties of a fluorophore in the vicinity of the metal surface may be used to develop novel assays and sensors. In one example the nanoparticle may be labeled with one member of a specific binding pair (antibody, protein, receptor etc) and the complementary member (antigen, ligand) may be labeled with a fluorescent molecule in such a way that the interaction of both binding partners leads to an detectable change in one or more fluorescence properties (such as intensity, quantum yield, lifetime, among others). Replacement of the labeled binding partner from the metal surface may lead to a change in fluorescence, that can then be used to detect and/or quantify an analyte.

Gold colloids can be synthesized by citrate reduction of a diluted aqueous HAuCl₄ solution. These gold nanoparticles are negatively charged due to chemisorption of citrate ions. Surface functionalization may be achieved by reacting the nanoparticles with thiolated linker groups containing amino or carboxy functions. In another approach, thiolated biomolecules are used directly for coupling to these particles.

In recent studies (T. Fare et al., Anal. Chem. 75 (17), 4672-4675, 2003) researchers made an observation that the fluorescence signals of cyanine dyes such as CY5 dye and the ALEXA 647 dyes in microarrays are strongly dependent on the concentration of ozone during posthybridization array washing. Controlled exposures of microarrays to ozone confirmed this factor as the root cause, and showed the susceptibility of a class of cyanine dyes (e.g., CY5 dyes, ALEXA 647 dyes) to ozone levels as low as 5-10 ppb for periods as short as 10-30 s.

One of the significant findings was the low dose level (ozone concentration multiplied by exposure time) that could induce the onset of the phenomenon, suggesting many labs may be at risk. For example, it is not uncommon that the environmental ozone levels would exceed 60 ppb during peak traffic hours on a sunny summer afternoon. Reporter compounds present on or in arrays that are exposed to these levels for as short as 1 min may begin to show significant degradation in a typical laboratory setting.

There are ways that help to eliminate the occurrence of ozone effects on microarrays, for example by equipping laboratories with HVAC systems having filters to significantly reduce ozone levels, or the use of dye-protecting solutions to avoid signal degradation. However, each of these approaches may add additional costs and/or time to perform the assay. These findings suggest the need for dyes and labels in the 600 to 700 nm wavelength range with improved chemical and photochemical stability.

Experimental data on cyanine dyes indicate that introduction of electron-withdrawing groups into the dye backbone may increase the photostability of such dyes. In addition it has been found that ring-substitution of squaraine dyes in the central squaraine ring with electron-withdrawing groups may lead to dyes with exceptional phototostabilities.

Analytes

The disclosed compositions may be used to detect an analyte that interacts with a recognition moiety in a detectable manner. As such, the compounds can be attached to a recognition moiety which is known to those of skill in the art. Such recognition moieties allow the detection of specific analytes. Examples are pH-, or potassium sensing molecules, e.g., synthesized by introduction of potassium chelators such as crown-ethers (aza crowns, thia crowns etc). Dyes with N-H substitution in the heterocyclic rings are known to exhibit pH-sensitive absorption and emission (S. Miltsov et al., Tetrahedron Lett. 40: 4067-68, (1999), M. E. Cooper et al., J. Chem. Soc. Chem. Commun. 2000, 2323-2324), Calcium-sensors based on the BAPTA (1,2-Bis(2-aminophenoxy)ethan-N,N,N′,N′-tetra-acetic acid) chelating moiety are frequently used to trace intracellular ion concentrations. The combination of a compound of the disclosure and the calcium-binding moiety BAPTA may lead to new long-wavelength absorbing and emitting Ca-sensors which could be used for determination of intra- and extracellular calcium concentrations (Akkaya et al. Tetrahedron Lett. 38:4513-4516 (1997). Additionally, or in the alternative, reporter compounds already having a plurality of carboxyl functional groups may be directly used for sensing and/or quantifying physiologically and environmentally relevant ions.

Fluorescence Methods

When fluorescent, the disclosed reporter compounds may be detected using common intensity-based fluorescence methods. These dyes are known to have lifetimes in the range of hundreds of ps to a few ns. The nanosecond lifetime and long-wavelength absorption and emission of these dyes when bound to proteins may allow them to be measured using relatively inexpensive instrumentation that employs laser diodes for excitation and avalanche photodiodes for detection. Typical assays based on the measurement of the fluorescence lifetime as a parameter include for example FRET (fluorescence resonance energy transfer) assays. The binding between a fluorescent donor labeled species (typically an antigen) and a fluorescent acceptor labeled species may be accompanied by a change in the intensity and the fluorescence lifetime. The lifetime can be measured using intensity- or phase-modulation-based methods (J. R. LAKOWICZ, PRINCIPLES OF FLUORESCENCE SPECTROSCOPY (2^{nd} Ed. 1999)).

Specific dyes of this disclosure may be used as non-fluorescent acceptor dyes for covalent labeling of molecular beacons, peptides and oligo probes for real-time PCR and FRET applications. Selected compounds of this disclosure (e.g. 5b, see Table below) have much higher extinction coefficients than conventional non-fluorescent quenchers that are commercially available (e.g. Invitrogen's QSY dyes or the Black Hole Quenchers™ from Biosearch Technologies), and therefore they are excellent candidates for use as non-fluorescent quenchers in FRET based applications.

| Compound | ε [Mol⁻¹ · cm⁻¹] |
|---|---|
| 5b (this disclosure) | 200,000 |
| QSY dyes (Invitrogen) | 23,000-90,000 |
| BHQ ™ | 34,000-43,000 |

Cyanine dyes exhibit high intrinsic polarization in the absence of rotational motion, making them useful as tracers in fluorescence polarization (FP) assays. Fluorescence polarization immunoassays (FPI) are widely applied to quantify low molecular weight antigens. The assays are based on polarization measurements of antigens labeled with fluorescent probes. The requirement for polarization probes used in FPIs is that emission from the unbound labeled antigen be depolarized and increase upon binding to the antibody. Low molecular weight species labeled with the compounds of the present disclosure can be used in such binding assays, and the unknown analyte concentration can determined by the change in polarized emission from the fluorescent tracer molecule.

Compositions and Kits

The present disclosure also provides compositions, kits and integrated systems for practicing the various aspects and embodiments of the invention, including producing the novel compounds and practicing of assays. Such kits and systems may include a reporter compound as described above, and may optionally include one or more of solvents, buffers, calibration standards, enzymes, enzyme substrates, and additional reporter compounds having similar or distinctly different optical properties.

Although the invention has been disclosed in preferred forms, the specific embodiments thereof as disclosed and illustrated herein are not to be considered in a limiting sense, because numerous variations are possible. Applicant regards the subject matter of his invention to include all novel and nonobvious combinations and subcombinations of the various elements, features, functions, and/or properties disclosed herein. No single element, feature, function, or property of the disclosed embodiments is essential. The following claims define certain combinations and subcombinations of elements, features, functions, and/or properties that are regarded as novel and nonobvious. Other combinations and subcombinations may be claimed through amendment of the present claims or presentation of new claims in this or a related application. Such claims, whether they are broader, narrower, or equal in scope to the original claims, also are regarded as included within the subject matter of applicant's invention.

We claim:

1. A composition of matter comprising a reporter compound according to the formula: 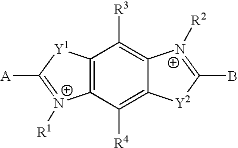
wherein
A is selected from the group consisting of H, alkyl, alkenyl, alkynyl, aryl, halogen, sulfo, carboxy, formylmethylene, phosphate, amino, sulfate, phosphonate, cyano, nitro, azido, reactive aliphatic and reactive aromatic groups and W¹, W², W³, W⁴, W⁵; wherein W¹, W², W³, W⁴, W⁵ have the respective formulae: 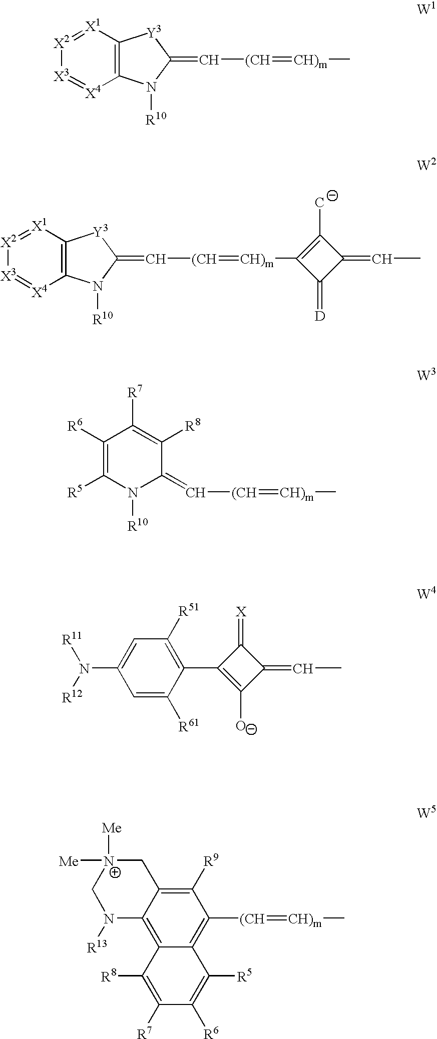
B is selected from the group consisting of W¹, W², W³, W⁴, W⁵;
each R¹, R² and R¹⁰ is independently selected from H, aliphatic groups, alicyclic groups, alkylaryl groups, aromatic groups, -L-S_{c}, -L-R^{x}, -L-R^{±}, —CH₂—CONH—SO₂-Me; each aliphatic residue may incorporate up to six heteroatoms selected from N, O, S, and can be substituted one or more times by F, Cl, Br, I, hydroxy, alkoxy, carboxy, sulfo, phosphate, amino, sulfate, phosphonate, cyano, nitro, azido, alkyl-amino, dialkyl-amino or trialkylammonium;
L is a covalent linkage that is linear or branched, cyclic or heterocyclic, saturated or unsaturated, having 1-20 nonhydrogen atoms from the group of C, N, P, O and S, in such a way that the linkage contains any combination of ether, thioether, amine, ester, amide bonds; single, double, triple or aromatic carbon-carbon bonds; or carbon-sulfur bonds, carbon-nitrogen bonds, phosphorus-sulfur, nitrogen-nitrogen, nitrogen-oxygen or nitrogen-platinum bonds, or aromatic or heteroaromatic bonds;
R^{x} is a reactive group;
S_{c} is a conjugated substance;
R^{±} is an ionic group;
each of X¹, X², X³, and X⁴ are independently selected from the group consisting of N, NR^{ι}, O, S, and C—R^{τ}, where R^{ι} is hydrogen, alkyl, arylalkyl and aryl groups, -L-S_{c}, -L-R^{x}, -L-R^{±}, —CH₂—CONH—SO₂-Me, where each aliphatic residue may incorporate up to six heteroatoms selected from N, O, S, and can be substituted one or more times by F, Cl, Br, I, hydroxy, alkoxy, carboxy, sulfo, phosphate, amino, sulfate, phosphonate, cyano, nitro, azido, alkyl-amino, dialkyl-amino or trialkylammonium;
R^{τ}, R³, R⁴, R⁵, R⁶, R⁷, R⁸ and R⁹ are each independently hydrogen, -L-S_{c}, -L-R^{x}, -L-R^{±}, —R^{x}, —R^{±}, —CH₂—CONH—SO₂-Me, amino, alkylamino, dialkylamino, trialkylammonium, sulfo, carboxy, nitro, cyano, azido, trifluoromethyl, alkoxy, halogen, carboxy, hydroxy, phosphate, sulfate or an aliphatic, alicyclic, or aromatic group; each aliphatic residue may incorporate up to six heteroatoms selected from N, O, S, and can be substituted one or more times by F, Cl, Br, I, hydroxy, alkoxy, carboxy, sulfo, phosphate, amino, sulfate, phosphonate, cyano, nitro, azido, alkyl-amino, dialkyl-amino or trialkylammonium;
or adjacent R^{ι}, R^{τ}, R⁵, R⁶, R⁷ and R⁸ substituents, when taken in combination, form a fused aromatic or heterocyclic ring that is itself optionally further substituted by H, alkyl, aryl, cycloalkyl, L-S_{c}, L-R^{x}, L-R^{±}, —R^{x} or —R^{±};
Y¹; Y² and Y³ are each independently selected from O, S, Se, N—R^{d}, CR^{e}═CR^{f} and C(Rⁱ)(R^{j}), wherein R^{d} is selected from the group consisting of H, aliphatic groups, alicyclic groups, aromatic groups, -L-S_{c}, -L-R^{x}, -L-R^{±}, —CH₂—CONH—SO₂-Me; and R^{e}, R^{f}, Rⁱ and R^{j} are selected from the group consisting of H, aliphatic groups, alicyclic groups, aromatic groups, -L-S_{c}, -L-R^{x}, -L-R^{±}, —R^{x}, —R^{±}, —CH₂—CONH—SO₂-Me, —COOH, —CN, —OH, —SO₃H, —PO₃H₂, —O—PO₃H₂, —PO₃R₂^{m}, —O—PO₃R₂^{m}, —CONHR^{m}, —CONH₂, COO—NHS and COO—R^{m}; each aliphatic residue may incorporate up to six heteroatoms selected from N, O, S, and can be substituted one or more times by F, Cl, Br, I, hydroxy, alkoxy, carboxy, sulfo, phosphate, amino, sulfate, phosphonate, cyano, nitro, azido, alkyl-amino, dialkyl-amino or trialkylammonium; R^{m} is selected from a group consisting of aliphatic groups, —(CH₂)_{y}—S_{c}, —(CH₂)_{y}—R^{x}, —(CH₂)_{y}—R^{±}, —(CH₂)_{y}—O—(CH₂)_{y}—S_{c}, —(CH₂)_{y}—O—(CH₂)_{y}—R^{x}, —(CH₂)_{y}—O—(CH₂)_{y}—R^{±}, where y is 1 to 20; and aromatic substituents; or Rⁱ and R^{j} taken in combination form a ring-system that is optionally further substituted by one or more reactive or ionic substituents;
D when present and neutral, is selected from the group consisting of ═O, ═S, ═Se, ═Te, ═N—R^{a}, and ═C(R^{b})(R^{c});
C when present and negatively charged, is selected from the group consisting of —O⁻, —S⁻, —Se⁻, —Te⁻, —(N—R^{a})⁻, and —(C(R^{b})(R^{c}))⁻; C can also be selected from —(N(R^{d})(R^{e})), in which case C is neutral.
each R^{a} may be independently selected from the group consisting of H, aliphatic, aromatic, alicyclic, aryl-alkyl, linked carriers, reactive and reactive aliphatic substituents, —COOH, —CN, —OH, —SO₃H, —SO₃R^{m}, —PO₃H₂, —O—PO₃H₂, —PO₃R₂^{m}, —O—PO₃R₂^{m}, —CONHR^{m}, —CONH₂, COO—NHS and COO—R^{m}; each aliphatic residue may incorporate up to six heteroatoms selected from N, O, S, and can be substituted one or more times by F, Cl, Br, I, hydroxy, alkoxy, carboxy, sulfo, phosphate, amino, sulfate, phosphonate, cyano, nitro, azido, alkyl-amino, dialkyl-amino or trialkylammonium; R^{m} is selected from a group consisting of aliphatic groups, —(CH₂)_{y}—S_{c}, —(CH₂)_{y}—R^{x}, —(CH₂)_{y}—R^{±}, where y is 1 to 20, and aromatic substituents;
each R^{b} and R^{c} may be independently selected from the group consisting of H, aliphatic, aromatic, alicyclic, aryl-alkyl, -L-S_{c}, -L-R^{x}, -L-R^{±}, —COOH, —CN, —OH, —SO₃H, —PO₃H₂, —O—PO₃H₂, —PO₃R₂^{m}, —O—PO₃R₂^{m}, —CONHR^{m}, —CONH₂, COO—NHS and COO—R^{m};
each aliphatic residue may incorporate up to six heteroatoms selected from N, O, S, and can be substituted one or more times by F, Cl, Br, I, hydroxy, alkoxy, carboxy, sulfo, phosphate, amino, sulfate, phosphonate, cyano, nitro, azido, alkyl-amino, dialkyl-amino or trialkylammonium;
each R^{d} and R^{e} may be independently selected from the group consisting of H, aliphatic, aromatic, alicyclic, aryl-alkyl, -L-S_{c}, -L-R^{x}, -L-R^{±}; each aliphatic residue may incorporate up to six heteroatoms selected from N, O, S, and can be substituted one or more times by F, Cl, Br, I, hydroxy, alkoxy, carboxy, sulfo, phosphate, amino, sulfate, phosphonate, cyano, nitro, azido, alkyl-amino, dialkyl-amino or trialkylammonium; R^{m} is selected from a group consisting of aliphatic groups, —(CH₂)_{y}—S_{c}, —(CH₂)_{y}—R^{x}, —(CH₂)_{y}—R^{±}, —(CH₂)_{y}—O—(CH₂)_{y}—S_{c}, —(CH₂)_{y}—R^{x}, —(CH₂)_{y}—O—(CH₂)_{y}—R^{±}, where y is 1 to 20, and aromatic substituents;
or R^{b} and R^{c}, taken in combination, form a cyclic or heterocyclic ring structure which is optionally substituted by -L-S_{c}, L-R^{x} or -L-R^{±};
R¹¹, R¹² and R¹³ are independently H, alkyl, aryl, -L-S_{c}, -L-R^{x}, -L-R^{±}, or taken in combination, form a cyclic or heterocyclic ring structure which is optionally substituted by -L-S_{c}, L-R^{x} or -L-R^{±};
R⁵¹ and R⁶¹ are independently H, OH, O-alkyl, NH-alkyl, NH-aryl;
m is 0, 1, 2 or 3;
and each H may be independently replaced by a fluorine.

2. The composition of claim 1, wherein at least one substituent includes a reactive group R^{x}.

3. The composition of claim 2, wherein the reactive group R^{x} is selected for reacting with amine moieties from the group consisting of N-hydroxysuccinimide esters, isothiocyanates, and sulfonylhalogenides.

4. The composition of claim 2, wherein the reactive group R^{x} is selected for reacting with thiol moieties from the group consisting of iodoacetamides and maleimides.

5. The composition of claim 2, wherein the reactive group R^{x} is selected for reacting with nucleic acids from the group consisting of phosphoramidites.

6. The composition of claim 1, wherein at least one substituent includes a linked carrier L-S_{c}.

7. The composition of claim 6, wherein the carrier S_{c} is selected from the group consisting of polypeptides, polynucleotides, beads, microplate well surfaces, lipids, small-molecule drugs, lectins, pharmacological agents and metallic nanoparticles.

8. The composition of claim 7, wherein the carrier S_{c} is a polypeptide or a polynucleotide.

9. The composition of claim 8, wherein the carrier S_{c} is a protein or DNA.

10. The composition of claim 1, further comprising a carrier S_{c}, which is associated covalently with the reporter compound through reaction with a reactive group on at least one substituent.

11. The composition of claim 1, wherein at least one substituent is R^{±} capable of increasing the hydrophilicity of the entire compound.

12. The composition of claim 11, wherein the R^{±} substituent is selected from the group consisting of —CH₂—CONH—SO₂-Me, SO₃⁻, COO⁻, PO₃²⁻, O—PO₃²⁻, PO₃R⁻, O—PO₃R⁻ and N(R^{ι})₃⁺, wherein R and R^{ι} are independently an aliphatic or aromatic moiety.

13. The composition of claim 1, wherein the substituents are selected so that the reporter compound is electrically neutral, increasing its hydrophobicity.

14. The composition of claim 1, wherein the substituents are selected so that the reporter compound contains a positive or negative net charge that increases its solubility in aqueous media and reduces its aggregation tendency in water and/or when covalently bound to proteins or other biomolecules.

15. The composition of claim 1, wherein the reporter compound is capable of covalently reacting with at least one of biological cells, DNA, lipids, nucleotides, polymers, proteins, lectins, pharmacological agents and solid surfaces.

16. The composition of claim 1, wherein the reporter compound is covalently or noncovalently associated with at least one of biological cells, DNA, lipids, nucleotides, polymers, proteins, and pharmacological agents.

17. The composition of claim 1, wherein m is 0.

18. The composition of claim 1, further comprising a second reporter compound selected from the group consisting of luminophores and chromophores.

19. The composition of claim 18, wherein one of the reporter compound and the second reporter compound is an energy transfer donor and the other is a corresponding energy transfer acceptor.

20. The composition of claim 18, wherein one of the first and second reporter compounds is an energy transfer acceptor and the other of the first and second reporter compounds is a corresponding energy transfer donor.

21. A composition of claim 1 further including a metallic nanoparticle that is selected to influence the photophysical properties of the reporter compound at a selected distance.

22. The composition of claim 21, wherein binding between the dye-conjugate and the nanoparticle is facilitated via a specific binding pair.

23. The claim of 22, wherein the specific binding pair is selected from the group consisting of antigens and antibodies, ligands and receptors, biotin and streptavidin, lectin and sugar, protein A and antibodies, and oligonucleotides and complementary oligonucleotides.

24. The composition of claim 1, comprising a compound having the following formula: 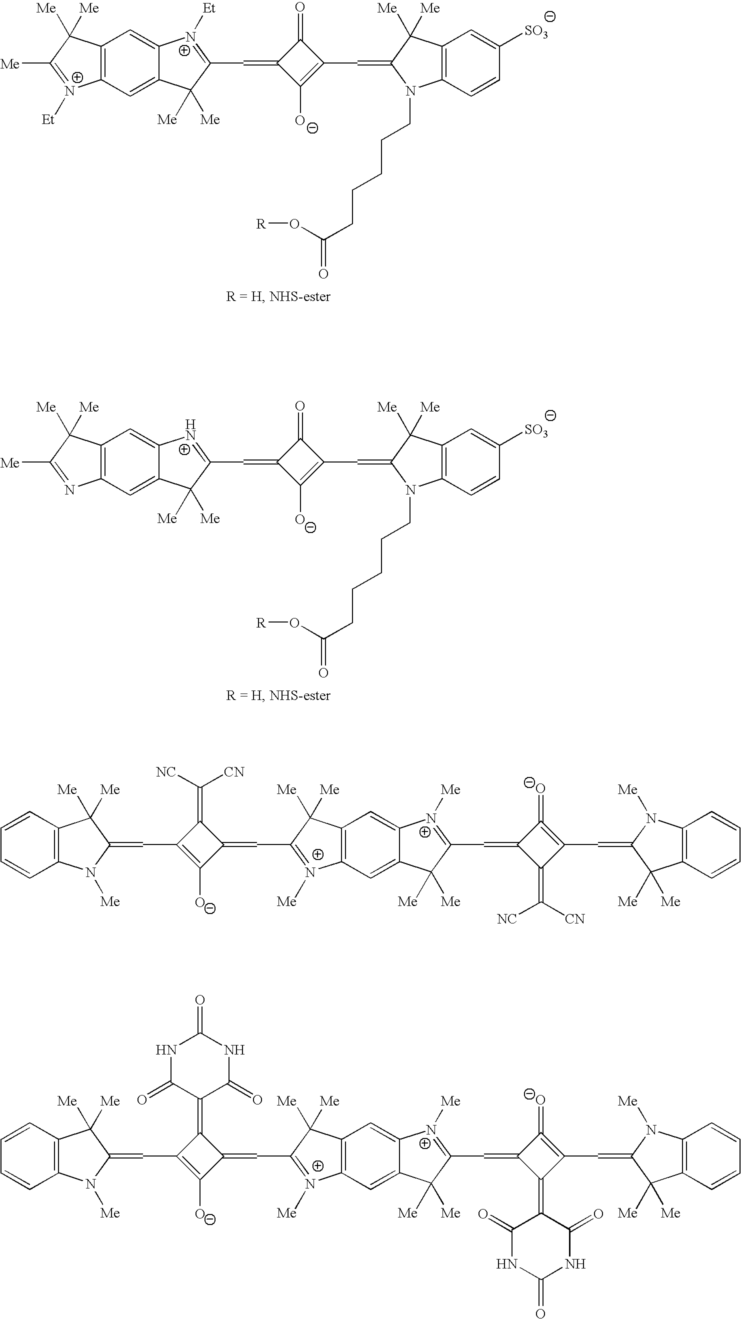 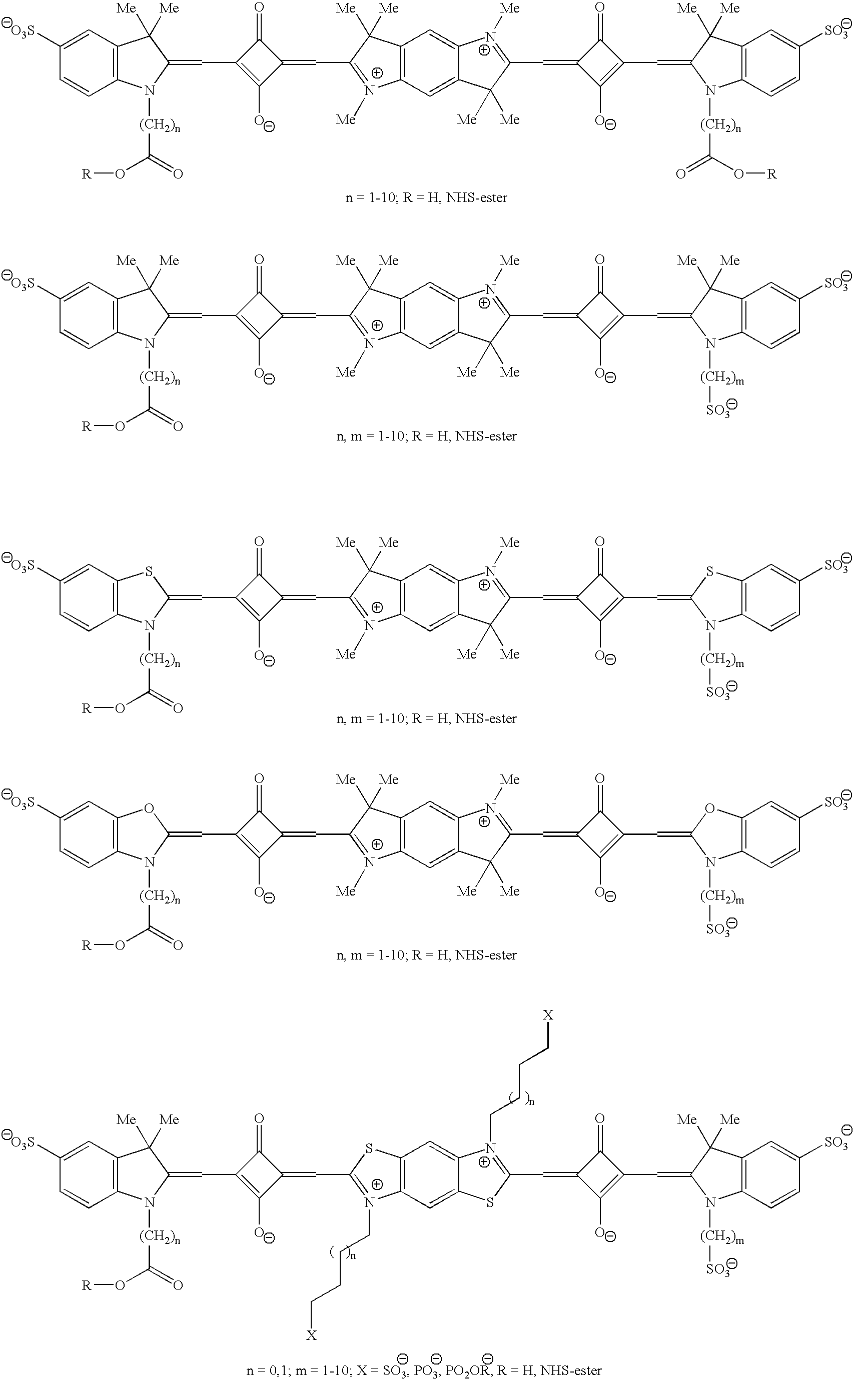 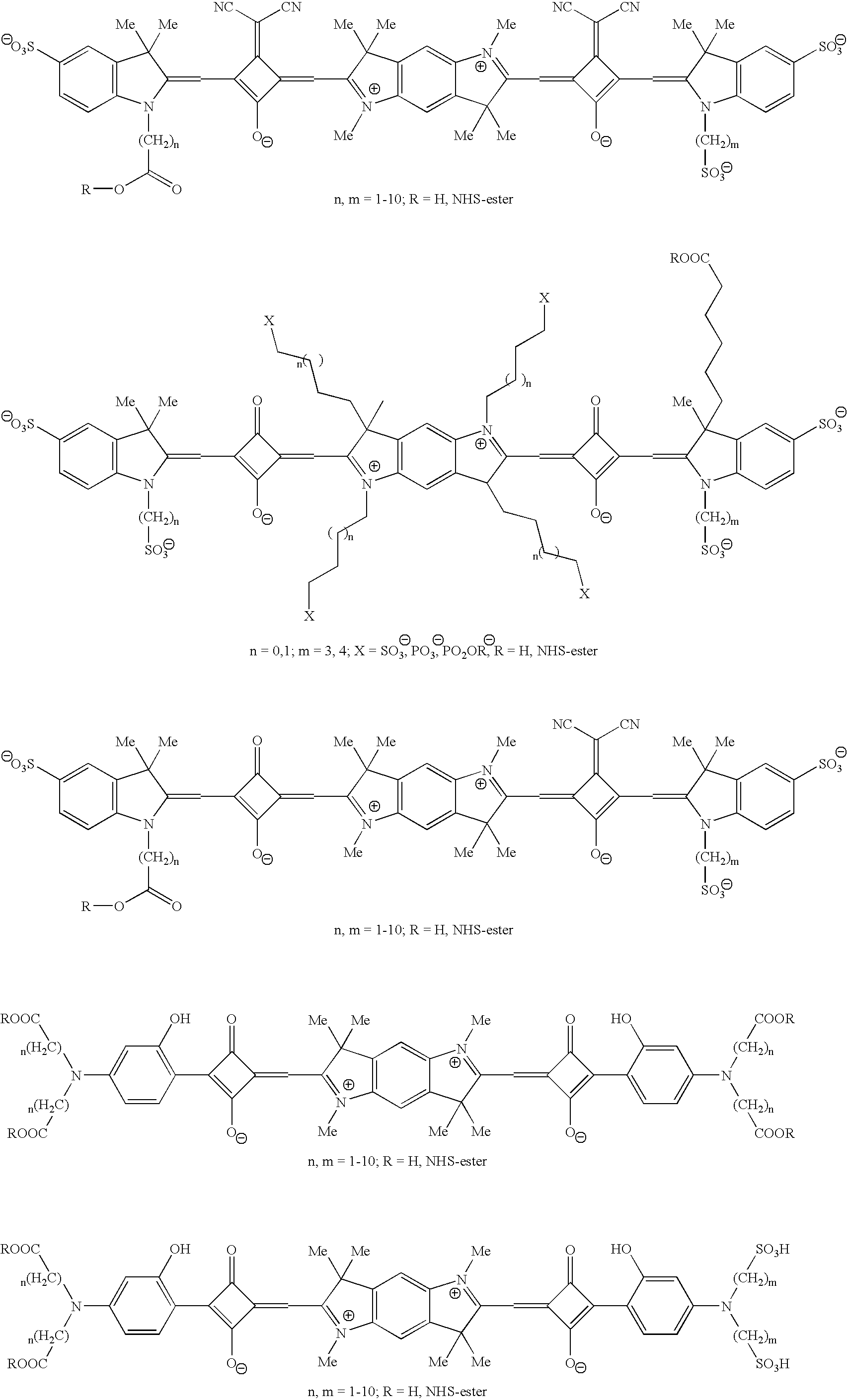 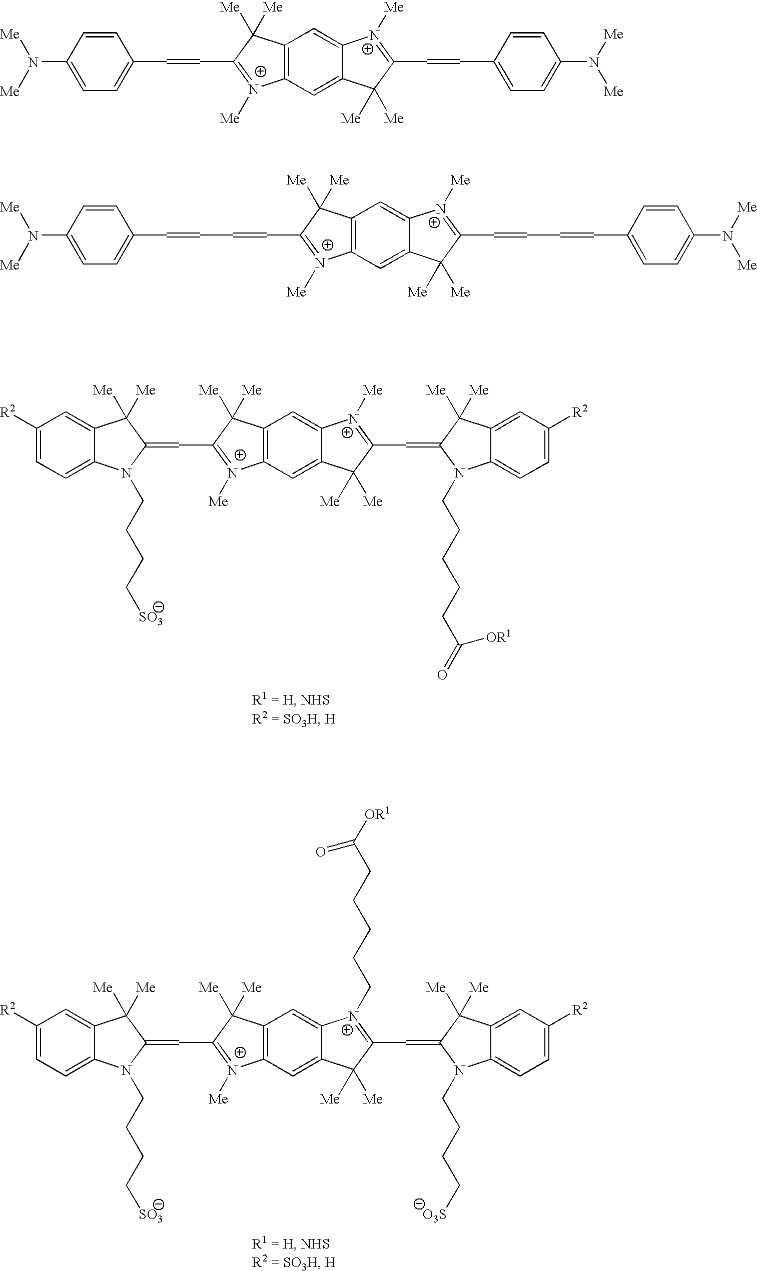 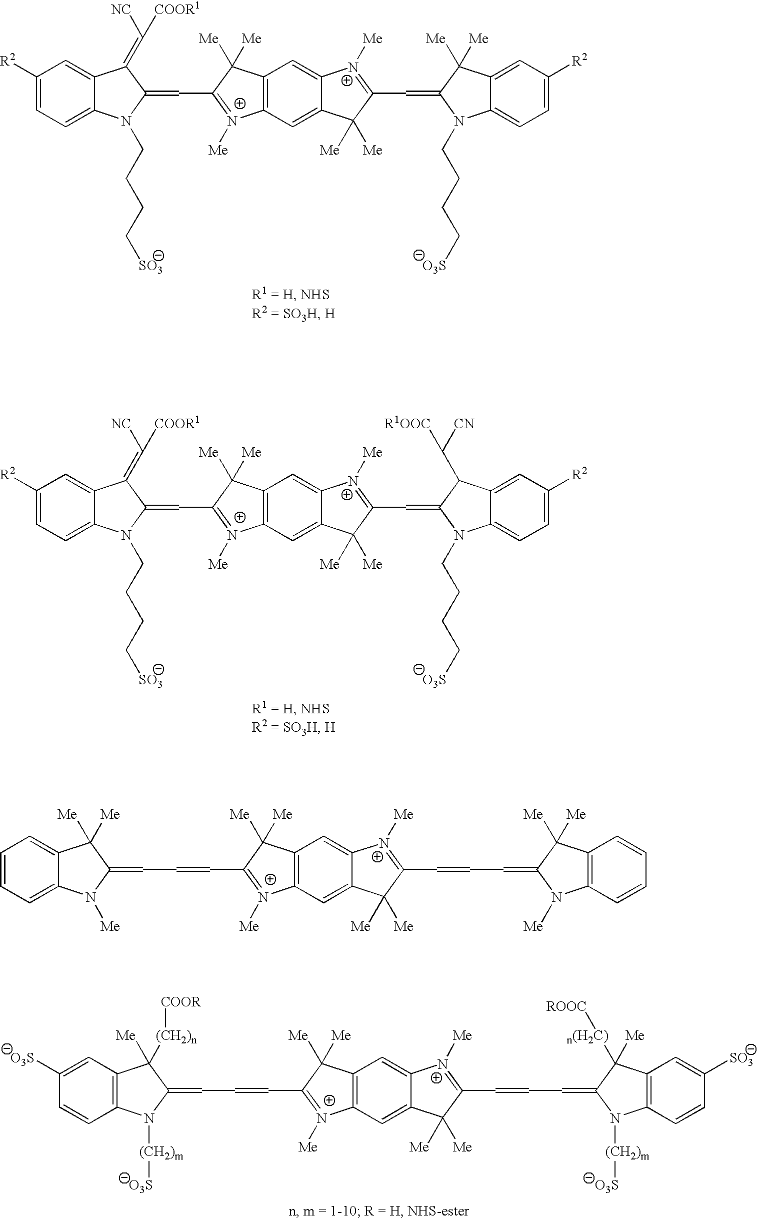 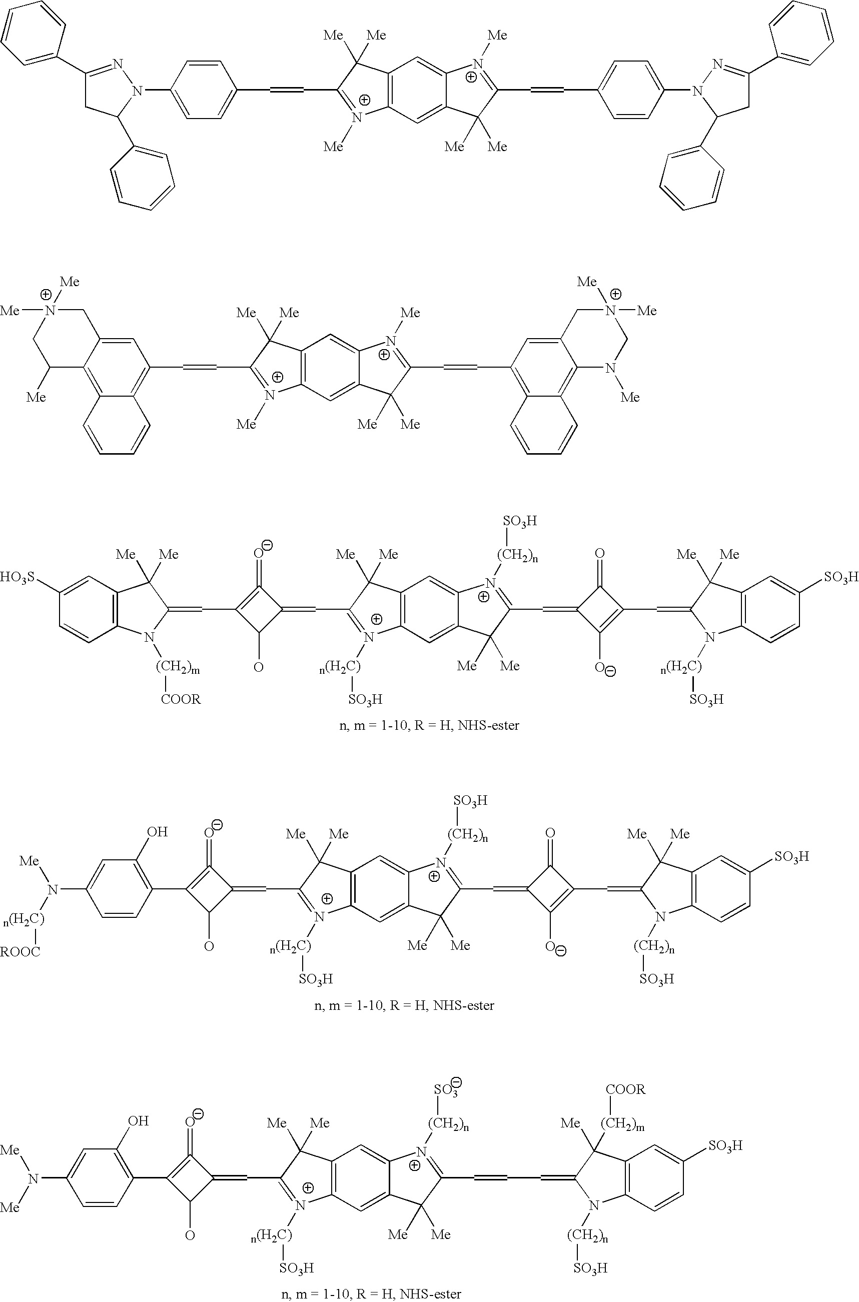 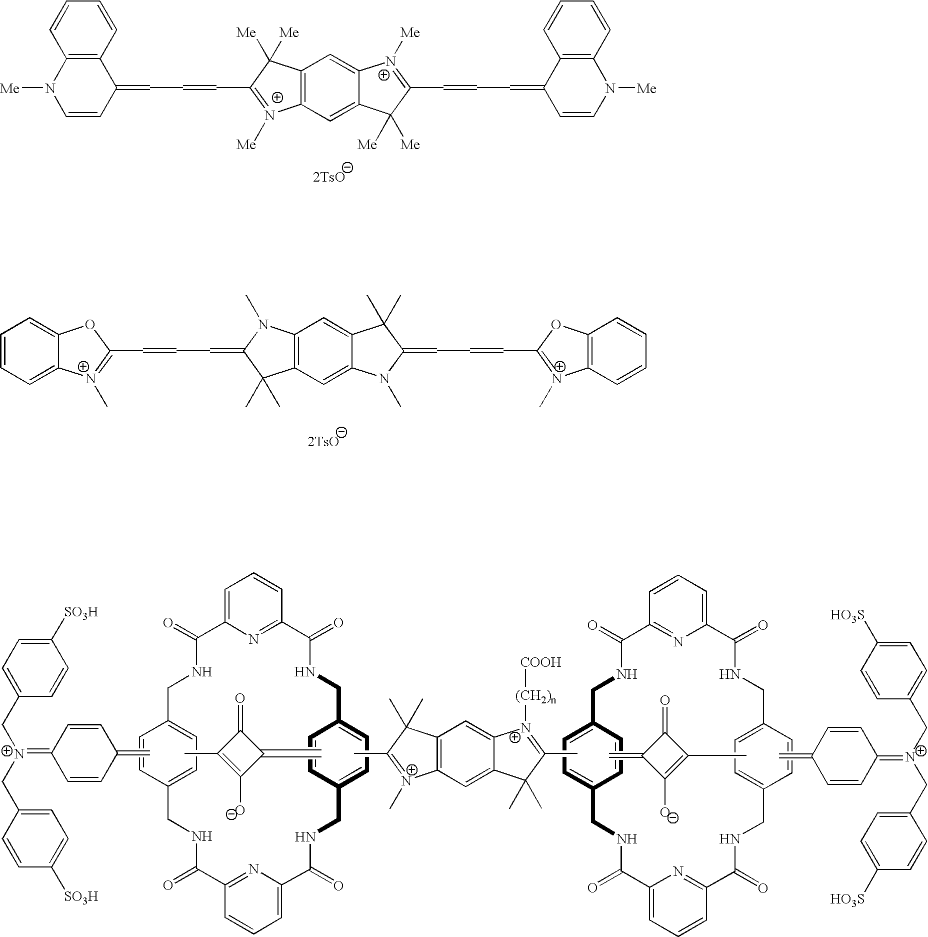
wherein the COOH group can be converted to NHS esters or is replaced by other reactive groups such as maleimide, iodoacetamide, among others. 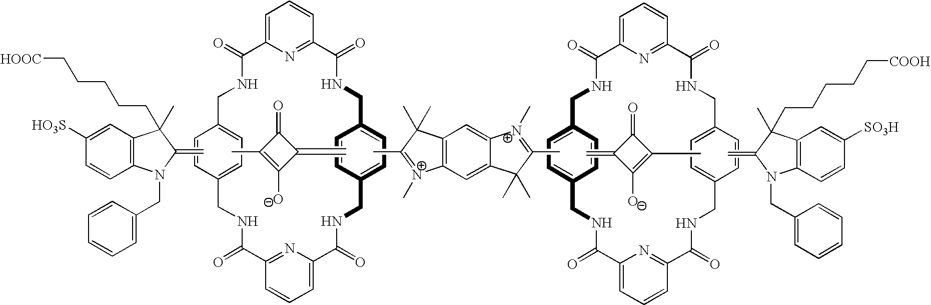
wherein the COOH group can be converted to NHS esters or is replaced by other reactive groups such as maleimide, iodoacetamide, among others. 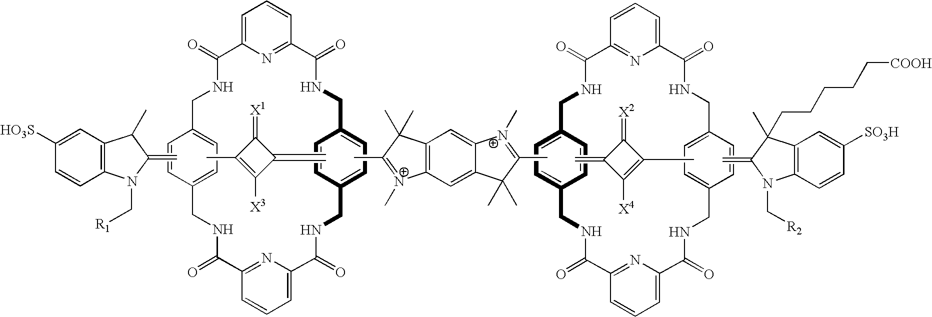
wherein X¹ and X², are independently O, S, C(CN)₂, N—R, where R is alkyl; X³ and X⁴ are independently O⁻, S⁻; R₁ and R₂ are alkyl, sulfo-alkyl, alkyl-phosphate, alkyl-phosphonate; the COOH group can be converted to NHS esters or is replaced by other reactive groups such as maleimide, iodoacetamide, among others. 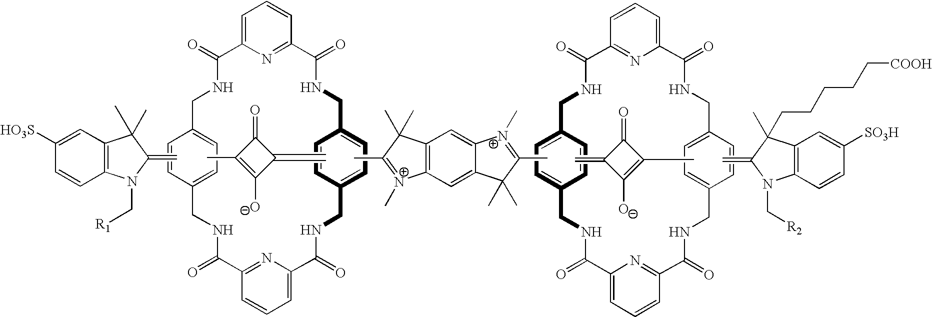
wherein R₁ and R₂ are alkyl, sulfo-alkyl, alkyl-phosphate, alkyl-phosphonate, among others; the COOH group can be converted to NHS esters or is replaced by other reactive groups such as maleimide, iodoacetamide, among others.
